(19) United States
(12) Patent Application Publication
(10) Pub. No.: US 2016/0115547 A1
(43) Pub. Date: Apr. 28, 2016

(54) USE OF FREE CIRCULATING DNA FOR DIAGNOSIS, PROGNOSIS, AND TREATMENT OF CANCER

(71) Applicant: JOHN WAYNE CANCER INSTITUTE, SANTA MONICA, CA (US)

(72) Inventors: Dave S.B. Hoon, Santa Monica, CA (US); Naoyuki Umetani, Tokyo (JP); Eiji Sunami, Los Angeles, CA (US)

(21) Appl. No.: 14/752,609

(22) Filed: Jun. 26, 2015

Related U.S. Application Data
(63) Continuation of application No. 11/915,711, filed on Sep. 29, 2008, now abandoned.
(60) Provisional application No. 60/685,148, filed on May 27, 2005.

(51) Int. Cl. C12Q 1/68

(52) U.S. Cl. CPC C12Q 1/6886; C12Q 1/6806

(57) ABSTRACT

A method of detecting circulating DNA in a body fluid. The method comprises identifying a subject suffering from or at risk for developing cancer, obtaining a body fluid sample from the subject, and determining the sequence integrity of circulating DNA in the sample, wherein the circulating DNA is not purified from the sample.

### RELATED APPLICATION

This application claims priority to U.S. Provisional Application Ser. No. 60/685,148, filed May 27, 2005, the content of which is incorporated herein by reference in its entirety.

This invention was made with support in part by grants from the National Cancer Institute (P01 CA 29605 and P01 CA 12582). Therefore, the U.S. government has certain rights.

### FIELD OF THE INVENTION

The present invention relates in general to cancer. More specifically, the invention relates to the use of free circulating DNA as a marker for diagnosis, prognosis, and treatment of cancer.

### BACKGROUND OF THE INVENTION

The use of genomic biomarkers as surrogates of tumor recurrence can permit the characterization of aggressive tumors, but improvement is needed, particularly in assay sensitivity, prognostic utility, and methods of serial sampling. Blood tests address these criteria in that they are minimally invasive, cost effective, and amenable to frequent analyses. Tumor cells have been shown to release DNA into body fluids such as blood (1-11). As a result, tumor-related DNA can be identified by DNA extraction and PCR assessment. Although the presence of DNA biomarkers in acellular plasma/serum has been investigated, its relevance to tumor biology is unknown. There is a need to develop more robust assays and genotypic markers that can be related to functional tumor biology.

### SUMMARY OF THE INVENTION

This invention relates to methods for diagnosis, prognosis, and treatment of cancer using free circulating DNA in body fluids as a biomarker.

In one aspect, the invention features a method of detecting circulating DNA in a body fluid. The method comprises identifying a subject suffering from or at risk for developing cancer, obtaining a body fluid sample from the subject, and determining the sequence integrity of circulating DNA in the sample, wherein the circulating DNA is not purified from the sample.

By “DNA not purified from a body fluid sample” is meant that a body fluid sample is merely processed to eliminate cells (e.g., through centrifugation and/or filtration) and proteins (e.g., through proteinase K digestion), and that no further step is taken to purify the DNA from the sample.

In one embodiment, the circulating DNA includes a repetitive DNA marker sequence indicative of the sequence integrity of the circulating DNA. By “repetitive DNA sequence” is meant that there are multiple copies of the DNA sequence in the genome of an organism.

The integrity of the circulating DNA refers to the wholeness of the circulating DNA, including the sequence integrity and the methylation integrity. The sequence integrity refers to the completeness of a sequence. It may be indicated, e.g., by the total amount of the circulating DNA (i.e., the sum of the amount of the circulating DNA released from apoptotic cells and the amount of the circulating DNA released from cancer cells), the amount of the circulating DNA released from cancer cells, or the ratio of the amount of the circulating DNA released from cancer cells to the total amount of the circulating DNA. The methylation integrity refers to the completeness of the methylation of a sequence. It may be indicated, e.g., by the methylated status, the unmethylated status, or both, of the circulation DNA.

In one embodiment, the total amount of the circulating DNA is indicated by the amount of ALU115, the amount of the circulating DNA released from cancer cells is indicated by the amount of ALU247 or LINE1 297, and the ratio of the amount of the circulating DNA released from cancer cells to the total amount of the circulating DNA is indicated by the ratio of the amount of ALU247 to the amount of ALU115.

In another aspect, the invention features a method of detecting circulating DNA in a body fluid, comprising the steps of obtaining circulating DNA from a body fluid sample and detecting a combination of the sequence integrity and the methylation integrity of the circulating DNA in the sample.

In one embodiment, the methylation integrity of the circulating DNA is indicated by the unmethylated status of the circulating DNA. In particular, the unmethylated status of the circulating DNA may be indicated by the unmethylated status of a LINE1 sequence.

The invention also provides a method for diagnosis, prognosis, and treatment of cancer. The method comprises obtaining circulating DNA from a body fluid sample, detecting the methylation integrity of the circulating DNA in the sample using a LINE sequence as a marker, and applying the methylation integrity of the circulating DNA in diagnosis, prognosis, and treatment of cancer.

In some embodiments, the body fluid sample is from a subject identified to be suffering from or at risk for developing cancer.

Within the methods of the invention, a body fluid sample may be, e.g., a sample of serum, plasma, urine, saliva, bone marrow, lymphatic fluid, lacrimal fluid, serous fluid, peritoneal fluid, pleural fluid, ductal fluid from breast, gastric juice, or pancreatic juice. A cancer may be, e.g., a breast cancer, colorectal cancer (CRC), periampullary cancer (PAC), melanoma, prostate cancer (PCa), gastric cancer, leukemia/lymphoma, renal cell carcinoma, hepatocellular carcinoma, neural-derived tumor, head and neck cancer, lung cancer, or sarcoma.

The sequence integrity of the circulating DNA may be determined, e.g., using quantitative real-time polymerase chain reaction (qPCR), microarrays, probes by blotting, or gel electrophoresis based, colorimetric detection assays such as ELISA, chemiluminescence methods, digital detection, and mass spectrometry (MALDI-TOF). The methylation integrity of the circulating DNA may be detected and quantified, e.g., using quantitative analysis of methylated alleles (QAMA), qPCR, gel electrophoresis, microarrays, mass spectrometry, digital detection, or colorimetric based methods.

In some embodiments, the circulating DNA includes a short interspersed nuclear element (SINE) such as ALU, a long interspersed nuclear element (LINE) such as LINE1, or a combination thereof. Specifically, the circulating DNA may include ALU115, ALU247, LINE1 297, or a combination thereof.

ALU115 is an amplicon obtainable by amplifying the ALU repeats in the human genome using a forward primer 5′-CCTGAGGTCAGGAGTTCGAG-3′ (SEQ ID NO:1) and a reverse primer 5′-CCCGAGTAGCTGGGATTACA-3′ (SEQ ID NO:2). ALU115 has a size of 115 base pairs.

ALU247 is an amplicon obtainable by amplifying the ALU repeats in the human genome using a forward primer 5′-GTGGCTCACGCCTGTAATC-3′ (SEQ ID NO:3) and a reverse primer 5′-CAGGCTGGAGTGCAGTGG-3′ (SEQ ID NO:4). ALU247 has a size of 247 base pairs.

LINE1 297 is an amplicon obtainable by amplifying the LINE1 repeats in the human genome using a forward primer 5′-CAGATCAACGAGACAGAAAGTCA-3′ (SEQ ID NO:5) and a reverse primer 5′-TTCCCTCTACACACTGCTTTGA-3′ (SEQ ID NO:6). LINE1 297 has a size of 297 base pairs.

In one embodiment, the methylated LINE1 is detected and quantified using a forward primer 5′-GTCGAATAGGAATAGTTTCGG-3′ (SEQ ID NO:7) and a reverse primer 5′-ACTCCCTAACCCCTTACGCT-3′ (SEQ ID NO:8), and the unmethylated LINE1 is detected and quantified using a forward primer 5′-GTTGAATAGGAATAGTTTTGGTTT-3′ (SEQ ID NO:9) and a reverse primer 5′-ACTCCCTAACCCCTTACACTT-3′ (SEQ ID NO:10).

The invention provides simple, robust, highly-sensitive, and high-throughput methods for diagnosis, prognosis, and treatment of cancer. The above-mentioned and other features of this invention and the manner of obtaining and using them will become more apparent, and will be best understood, by reference to the following description, taken in conjunction with the accompanying drawings. These drawings depict only typical embodiments of the invention and do not therefore limit its scope.

### BRIEF DESCRIPTION OF THE FIGURES

FIG. 1. A consensus of human ALU interspersed repetitive sequence (SEQ ID NO:13). Primers of 115 bp amplicon are indicated by open boxes (SEQ ID NOS:1 and 11).

FIG. 2. A. PCR products of ALU primers on 10, 1, 0.1, and 0.01 pg (lanes 1, 2, 3, and 4, respectively) of genomic DNA templates by gel electrophoresis at 28 cycles of thermal cycling of qPCR. MM: molecular marker. B. Serially diluted genomic DNA (10 ng to 0.01 pg) obtained from PBL of a healthy volunteer was quantified by ALU-qPCR. Linearity was maintained in the 10⁶ range, and sensitivity was as low as 0.01 pg, equivalent to about 1/300 copy of genome in a single cell.

FIG. 3. ALU-qPCR quantification of DNA in serum (A) and plasma (B). The DNA concentration in serum and plasma samples was 970±730 pg/μl and 180±150 pg/μl (mean±SD), respectively.

FIG. 4. Distribution of serum:plasma DNA concentration ratios in pairs of specimens. A fitted normal distribution curve for all but one outlier pair which had a ratio of 32 (*) was overlapped.

FIG. 5. Scattergram of serum and plasma DNA concentrations and Deming's regression after square-root variable transformation to normalize the distribution of DNA concentrations. Deming regression model shown with a solid line was (serum DNA)^{0.5}=1.6×(plasma DNA)^{0.5}+8.7. The intercept value of serum DNA axis at (plasma DNA)=0 was 76 (95% CI: 7.8−210) pg/μl. The dotted line indicates the line of identity between serum and plasma.

FIG. 6. A. A consensus of human long-form ALU interspersed repetitive sequence (SEQ ID NO:13) with indication of the primers by open boxes [symbol] : ALU115 primers (115 bp amplicon) (SEQ ID NOS:1 and 11); solid underlines ______: ALU247 primers (247 bp amplicon) (SEQ ID NO:3 and 12). B. Calculated relative efficiency of DNA quantification in terms of DNA length. Estimated efficiency of ALU115 primers is shown as a solid line: 0% in <115 bp and 90% at 1150 bp; ALU247 primers as a dotted line: 0% in <247 bp and 90% at 2470 bp. Dotted area represents the size of DNA released from apoptotic cells.

FIG. 7. A. DNA integrity in serum from healthy females (N=51) and patients with pre-operative AJCC stage 0-IV breast cancer (N=8, 24, 27, 21, and 3, respectively). Bars on the right side indicate mean±SEM (standard error of the mean) values. Serum DNA integrity increased with AJCC stage (Spearman's ρ=0.54; P<0.0001). Serum DNA integrity was significantly higher in patients with pre-operative AJCC stage II-IV than in healthy females. B. ROC curve for discrimination of patients with AJCC stage II-IV breast cancer from healthy females by serum DNA integrity. AUC is 0.79 (95% CI: 0.70-0.86).

FIG. 8. Serum DNA integrity and tumor size of 75 invasive primary cancers were significantly correlated (R=0.48, P<0.0001). Non-invasive tumors (8 cases of DCIS) were not included in this analysis.

FIG. 9. Serum DNA integrity and LVI status in 82 patients with evaluable primary breast cancers.

FIG. 10. A. Serum DNA integrity and LN metastasis in 42 patients with LN metastasis and 41 patients without LN metastasis. B. ROC curve for discrimination of patients with LN metastasis by serum DNA integrity. AUC is 0.81 (95% CI: 0.72-0.89).

FIG. 11. A. A consensus sequence of interspersed human ALU repeats (SEQ ID NO:13). ALU115 primers (115 bp amplicon) are boxed (SEQ ID NOS:1 and 11); ALU247 primers (247 bp amplicon) are underlined (SEQ ID NOS:3 and 12). B. Calculated relative efficiency of DNA quantification in terms of DNA length. Estimated efficiency of ALU115 primers is shown as a solid line: 0% in <115 bp and 90% at 1150 bp; ALU247 primers as a dotted line: 0% in <247 bp and 90% at 2470 bp. The size of DNA released from apoptotic cells is indicated with arrows.

FIG. 12. A. Sensitivity and linearity of ALU-qPCR of serum DNA. Serially diluted genomic DNA (10 ng to 0.01 pg) was quantified by qPCR with ALU115 or ALU 247 primers. Linearity was maintained in 10⁶ range, and sensitivity was as low as 0.01 pg. B. Agarose gel view of PCR products of ALU115 and ALU247 primers on 10, 1, 0.1, and 0.01 pg (ALU115: lanes 1, 2, 3, and 4; ALU247: lanes 5, 6, 7, and 8, respectively) of genomic DNA templates with 28 cycles of qPCR. MM: molecular marker.

FIG. 13. DNA was conventionally extracted and purified from 500 μl of serum of 15 normal volunteers and 8 patients with PAC, and its amount was quantified by ALU-qPCR with ALU115 primers and PicoGreen method. A 1/10 and 1/5000 amount of total purified DNA (50 μl and 0.1 μl equivalent volume of serum) was used for each quantification by PicoGreen method and ALU qPCR method, respectively. Solid triangle on the vertical axis indicates the lower limit of PicoGreen method. Diagonal dotted line indicates assumed fit line of the two methods if they have no lower limits.

FIG. 14. A. Absolute levels of free circulating DNA in serum of normal volunteers and patients with stage I/II and stage III/IV CRC along with means diamonds indicating sample means and 95% confidence intervals. Patients with stage I/II and stage III/IV CRC had significantly higher absolute serum DNA level (P=0.006 and P=0.004, respectively). B. ROC curve for discriminating patients with stage I-IV CRC from healthy volunteers had an AUC of 0.75. Sensitivity was 0.41 at specificity of 0.90.

FIG. 15. A. Integrity of free circulating DNA in serum of normal volunteers and patients with stage I/II and stage III/IV CRC along with means diamonds indicating sample means and 95% confidence intervals. Patients with stage I/II and stage III/IV CRC had significantly higher serum DNA integrity (P=0.002 and P=0.006, respectively). B. ROC curve for discriminating patients with stage I-IV CRC from healthy volunteers had an AUC of 0.78. Sensitivity was 0.56 at specificity of 0.90.

FIG. 16. A. Absolute levels of free circulating DNA in serum of normal volunteers and patients with stage I/II and stage III/IV PAC along with means diamonds indicating sample means and 95% confidence intervals. No significant elevation of absolute serum DNA level was observed in patients with stage I/II and stage III/IV PAC. B. ROC curve for discriminating patients with stage I-IV PAC from healthy volunteers had an AUC of 0.59. Sensitivity was 0.31 at specificity of 0.90.

FIG. 17. A. Integrity of free circulating DNA in serum of normal volunteers and patients with stage I/II and stage III/IV PAC along with means diamonds indicating sample means and 95% confidence intervals. Patients with stage I/II and stage III/IV PAC had significantly higher serum DNA integrity (P=0.022 and P<0.0001, respectively). B. ROC curve for discriminating patients with stage I-IV PAC from healthy volunteers had an AUC of 0.80. Sensitivity was 0.58 at specificity of 0.90.

FIG. 18. A. Absolute amount of DNA quantified by qPCR with ALU247 primer set in normal and in AJCC stage 0/I, II, III, and IV melanomas. B. ROC curve for detection of stage 0-III melanomas (n=34) from normal individuals (n=65) by ALU247 qPCR. AUC was 0.87. C. ROC curve for detection of stage IV melanomas (n=49) from normal individuals (n=65) by ALU247 qPCR. AUC was 0.89.

FIG. 19. A. DNA integrity values in normal and in AJCC stage 0/I, II, III, and IV melanomas. B. ROC curve for detection of stage IV melanomas (n=49) from normal individuals (n=65) by DNA integrity. AUC was 0.82.

FIG. 20. A. Absolute amount of long DNA fragment quantified by qPCR with ALU247 primer set in normal group (age>40) and in stage IV prostate cancer patients. qPCR values of ALU247 in stage IV cancers were significantly higher than in normal group (P<0.0001). B. ROC curve for discrimination of stage IV prostate cancers from normal controls by ALU247 quantity (AUC=0.8256). C. A standard curve of qPCR analysis of LINE1 297.

FIG. 21. A. Representative capillary array electrophoresis (CAE) analysis: methylated and unmethylated LINE1 detection in prostate cancer patients' sera. B. Standard curve plot for the QAMA assay showing sensitivity of methylated LINE1 and unmethylated LINE1 (uLINE1) quantitative analysis. C. QAMA assay analysis curve of uLINE1 and methylated LINE1: cycle plot of cycle threshold to determine the cut-off value.

FIG. 22. A. Absolute amount of long DNA fragment quantified by qPCR with LINE297 primer set in normal group (age>40) and in stage IV prostate cancer patients. qPCR values of LINE297 in stage IV cancers were significantly higher than in normal group (P=0.0048). B. ROC curve for discrimination of stage IV prostate cancers from normal controls by LINE297 quantity (AUC=0.7211).

FIG. 23. The ratio of unmethylated LINE1 detection in normal group (n=23) and prostate cancer patient group (n=47). The unmethylated LINE1 was significantly higher in prostate cancer patients than in normal group (P=0.035, positive predictive value=0.85).

FIG. 24. The ratio of unmethylated LINE1 detection in normal group (n=23) and stage IV prostate cancer patient group (n=27). The unmethylated LINE1 was significantly higher in stage IV prostate cancer patients than in normal group (P=0.062, PPV=0.81, NPV=0.59).

FIG. 25. A. ROC curve for discrimination of prostate cancers from normal controls by combination of ALU247 quantity and LINE297 quantity (n=66, AUC=0.837). B. ROC curve for discrimination of prostate cancers from normal controls by combination of three markers (ALU247 quantity, LINE297 quantity, unmethylated LINE1 detection). AUC improves up to 0.912.

FIG. 26. Absolute amount of long DNA fragment quantified by qPCR with LINE297 primer set in normal group (female, n=29) and in breast cancer group (n=45). qPCR values of LINE297 in breast cancer patients were significantly higher than in normal group (P=0.045).

FIG. 27. Absolute amount of long DNA fragment quantified by qPCR with LINE297 primer set in lymph node (LN) negative group (n=27) and in LN positive group (n=18) of breast cancer patients. qPCR values of LINE297 of LN positive patients were significantly higher than those of LN negative patients (P=0.045).

FIG. 28. Absolute amount of long DNA fragment quantified by qPCR with LINE297 primer set in T0 to T1 group (n=27) and in T2 to T4 group (n=18) of breast cancer patients. qPCR values of LINE297 of T2 to T4 patients were significantly higher than those of T0 to T1 patients (P=0.047).

FIG. 29. Absolute amount of longer DNA Fragment quantified by qPCR with LINE297 primer set in AJCC stage 0-III breast cancers (n=45). qPCR values of LINE297 in stage III cancers were significantly higher than in stage 0 to I (P=0.015).

FIG. 30. A representative of LINE1 297 copy numbers for different AJCC stages of breast cancer.

### DETAILED DESCRIPTION OF THE INVENTION

The invention is based at least in part upon the unexpected discovery that ALU and LINE1 repeats in serum can be used as markers in simple, robust, highly-sensitive, and high-throughput methods for diagnosis, prognosis, and treatment of cancer.

Cell-free DNA circulating in body fluids is a molecular biomarker for malignant tumors. Unlike the uniformly truncated DNA released from apoptotic cells, DNA released from cancer cells due to necrosis, physical death, secretion, or disruption varies in size. Furthermore, altered methylation patterns of circulating DNA have been found to play a role in the development of cancer. Therefore, the sequence integrity and the methylation integrity of the circulating DNA are clinically useful for the detection and management of cancer.

Accordingly, the invention provides a method of detecting circulating DNA in a body fluid. The method involves identifying a subject suffering from or at risk for developing cancer, obtaining a body fluid sample from the subject, and determining the sequence integrity of circulating DNA in the sample, wherein the circulating DNA is not purified from the sample.

“Subject,” as used herein, refers to a human or animal, including all vertebrates, e.g., mammals such as primates (particularly higher primates), sheep, dog, rodents (e.g., mouse or rat), guinea pig, goat, pig, cat, rabbit, and cow, etc. In a preferred embodiment, the subject is a human. In another embodiment, the subject is an experimental animal or animal suitable as a disease model.

The method of the invention comprises a step of identifying a subject suffering from or at risk for developing cancer, with or without clinical evidence of the disease. For example, a subject for the method may be a subject having a family history of cancer, a pre-operative cancer patient, or a post-operative cancer patient. Subject identification can be in the judgment of the subject or a health care professional. It can be either subjective (e.g., opinion) or objective (e.g., measurable by a test or diagnostic method).

A body fluid sample contains any body fluid in which free circulating DNA released by cancer cells may be present. Examples of such body fluids include, without limitation, blood (serum/plasma), bone marrow (serum/plasma), cerebral spinal fluid, peritoneal fluid, pleural fluid, lymph fluid, ascites, serous fluid, sputum, lacrimal fluid, stool, urine, saliva, ductal fluid from breast, gastric juice, and pancreatic juice. Body fluids can be collected using any of the standard methods known in the art.

Purification of circulating DNA from a body fluid may cause loss of the DNA and contamination by DNA released from cells present in the body fluid. This usually results in a longer processing time, a complicated processing method, a higher cost, and lower sensitivity, specificity, and consistency. The method of the invention overcomes these problems by eliminating the unnecessary purification step. As described in the examples below, the potentially contaminating cells can be removed from a body fluid, e.g., by centrifugation and/or filtration. The proteins that may interfere with the detection of the circulating DNA can be removed, e.g., by proteinase K digestion. The minimally processed sample can then be used for detection of the circulating DNA without further purification.

Alternatively, the circulating DNA may be further purified after removal of the cells and proteins from the body fluid, using any of the methods known in the art. For example, the circulating DNA may be extracted with phenol, precipitated in alcohol, and dissolved in an aqueous solution.

The circulating DNA can be detected and quantified using a number of methods well known in the art, e.g., qPCR, microarrays, probes by blotting, or gel electrophoresis based, colorimetric detection assays such as ELISA, chemiluminescence methods, digital detection, and mass spectrometry (MALDI-TOF). In a preferred embodiment, qPCR is employed to allows routine and reliable quantification of PCR products.

In one embodiment, fluorogenic probes are used in qPCR. A fluorogenic probe is an oligonucleotide with both a reporter fluorescent dye and a quencher dye attached. While the probe is intact, the proximity of the quencher greatly reduces the fluorescence emitted by the reporter dye. During the extension phase of PCR, if a target sequence is present, the probe anneals downstream from one of the primer sites and is cleaved by the 5′ nuclease activity of Taq DNA polymerase as this primer is extended. This cleavage of the probe separates the reporter dye from the quencher dye, increasing the reporter dye signal. Cleavage removes the probe from the target strand, allowing primer extension to continue to the end of the template strand. Additional reporter dye molecules are cleaved from their respective probes with each cycle, effecting an increase in fluorescence intensity proportional to the amount of the amplicon produced.

Instruments for qPCR, e.g., ABI PRISM 7700 Sequence Detection System, are commercially available. Quantification of the amount of a target in a test samples is accomplished by measuring CT (cycle threshold) and using a standard curve to determine the starting copy number of the target in the sample.

The sensitivity of the method increases when repetitive DNA sequences are used as markers to indicate the sequence integrity of the circulating DNA. For example, the SINEs of primate-specific ALU sequences (approx. 300 bp) are the most abundant repetitive genomic sequences and account for >10% of the human genome, while LINE1s (>6 kb) represent about 17% of the human genome. As shown in the examples below, when free circulating DNA is quantified by qPCR for the ALU repeats, a sensitivity of 0.01 pg of DNA in serum can be achieved.

In healthy subjects, the main source of free circulating DNA is apoptotic cells. Apoptotic cells release DNA fragments that are usually 120-200 bp in length. By contrast, DNA released from cancer cells varies in size. The sequence integrity of the circulating DNA can be indicated by any mathematical representation involving the amount of the circulating DNA released from cancer cells. For example, the sequence integrity of the circulating DNA can be represented by the total amount of the circulating DNA, the amount of the circulating DNA released from cancer cells, or the ratio of the amount of the circulating DNA released from cancer cells to the total amount of the circulating DNA.

In one embodiment, to assess the total amount of the circulating DNA by qPCR, primers are designed such that they can be used to amplify a fragment in DNA released from both apoptotic cells and cancer cells undergoing non-apoptotic death. Typically, the size of the amplicon is in the range of 120-200 bp. Smaller fragments are rapidly cleared away from body fluids. In contrast, to assess the amount of the circulating DNA released from cancer cells but not apoptotic cells, primers are chosen to amplify a fragment present in the cancer cell-generated DNA but not the apoptotic cell-generated DNA. The size of such a fragment is generally in the range of 220-400 bp.

Also within the invention is a method of detecting circulating DNA in a body fluid, comprising the steps of obtaining circulating DNA from a body fluid sample and detecting a combination of the sequence integrity and the methylation integrity of the circulating DNA in the sample. Optionally, the method includes a step of identifying a subject suffering from or at risk for developing cancer, the subject being the source of the body fluid.

The sequence integrity of the circulating DNA (purified from a body fluid or not) may be detected and quantified using a variety of methods known in the art, including those described above.

Methods for extracting circulating DNA from a body fluid and determining the methylated/unmethylated status of a sequence are well known in the art. For example, circulating DNA can be extracted from a body fluid as described above. The methylated/unmethylated status of a sequence can be detected and quantified, for example, using QAMA, methylation-specific PCR, bisulfite sequencing (COBRA), pyrosequencing, qPCR, gel electrophoresis, microarrays, mass spectrometry, digital detection, or colorimetric based methods. In a preferred embodiment, QAMA is used to relatively quantify methylated and unmethylated alleles simultaneously amplified in a single reaction.

In one embodiment, the DNA is denatured and treated with bisulfite, converting all unmethylated, but not methylated, cytosines to uracils. After bisulfite modification, a methylated allele differs from an unmethylated allele at all CpG positions within the nucleotide sequence. Primers are designed to amplify the bisulfite-converted antisense strand of a target sequence. The primer binding sites lack CpG dinucleotides and, therefore, the methylated and unmethylated alleles can be amplified in the same reaction with one primer pair. Methylation discrimination occurs during hybridization of two differently labeled internal minor groove binder (MGB) TaqMan® probes, one specifically binding to the methylated allele, and the other specifically binding to the unmethylated allele. Probes bound to their respective target sites are cleaved by the 5′ nuclease activity of Taq DNA polymerase in the course of PCR, and the amplification of the methylated and unmethylated alleles is monitored independently. The amount of each fluorescent dye released during PCR is measured by a qPCR system and is directly proportional to the amount of the respective PCR product generated. Quantification of the methylated and unmethylated alleles is accomplished by comparison to standard curves.

The methylation integrity of the circulating DNA can be indicated by any mathematical representation involving the amount of the unmethylated allele. For example, the methylation integrity of the circulating DNA can be represented by the amount of the unmethylated allele, or the ratio of the amount of the unmethylated allele to the amount of the methylated allele.

The sensitivity of the method increases when repetitive DNA sequences are used as markers to indicate the methylation integrity of the circulating DNA. LINE1 is a particularly suitable marker as a non-coding and repetitive sequence. In a preferred embodiment, the methylation integrity of the circulating DNA is determined by assessing the unmethylated LINE1 only of a particular size.

When the integrity of the circulating DNA in a body fluid is found to be associated with a cancer parameter, the above methods of the invention can be used for diagnosis, prognosis, and treatment of cancer. Such association can be identified, e.g., from literature, by analysis of available data, or through studies as described in the examples below. For instance, in one embodiment, body fluid samples are collected from healthy controls and cancer patients or from cancer patients of different categories. The integrity of the circulating DNA is assessed, e.g., using the methods described above. The results for the controls and the patients or for the patients of different categories are compared. If the integrity of the circulating DNA is significantly different for the controls and the patients or for the patients of different categories, the integrity of the circulating DNA can be used as a marker for detection and management of cancer. In general, high amount/percentage of circulating DNA released from cancer cells and/or high amount/percentage of unmethylated circulating DNA in body fluids are associated with advanced cancers.

The examples below provide some cancer parameters associated with the integrity of the circulating DNA in serum, including the existence of cancer, the stage of cancer, the size of primary tumor, lymphovascular invasion, lymph node metastasis, and post-operative recurrence.

Specifically, in breast cancer, the serum ALU115-qPCR values are significantly higher in patients with AJCC stage II and III cancer than in healthy females, the serum ALU247-qPCR values are significantly higher in patients with stage II, III, and IV cancer than in healthy females, and the serum (ALU247-qPCR value)/(ALU115-qPCR value) ratios are significantly higher in patients with stage II, III, and IV cancer than in healthy females. In patients with invasive primary breast cancer, the serum (ALU247-qPCR value)/(ALU115-qPCR value) ratio and the size of primary tumor are significantly correlated. In patients with primary breast cancers, patients with lymphovascular invasion (LVI)-positive cancers have significantly higher serum (ALU247-qPCR value)/(ALU115-qPCR value) ratios than patients with LVI-negative cancers. In patients with primary breast cancer, patients with lymph node (LN) metastasis have significantly higher serum (ALU247-qPCR value)/(ALU115-qPCR value) ratios than patients without LN metastasis. The serum (ALU247-qPCR value)/(ALU115-qPCR value) ratio is a significant pre-operative predictor of LN metastasis. Patients with micrometastasis (pN1mi) have a serum (ALU247-qPCR value)/(ALU115-qPCR value) ratio significantly higher than that of LN-negative patients. Furthermore, patients with post-operative recurrence of breast cancer have a serum (ALU247-qPCR value)/(ALU115-qPCR value) ratio equivalent to that of the patients with stage III or IV primary breast cancer, and higher than that of healthy females. In addition, the serum LINE1 297-qPCR values are significantly higher in breast cancer patients than in normal group, in LN-positive group than in LN-negative group, in T2 to T4 group than in T0 to T1 group, and in stage III cancers than in stage 0 to I cancers.

In CRC, the serum ALU115-qPCR values in patients with stage I/II and III/IV CRC are significantly higher than in normal volunteers. The serum (ALU247-qPCR value)/(ALU115-qPCR value) ratios in patients with stage I/II and III/IV CRC are also significantly higher than in normal volunteers.

In PAC, the serum (ALU247-qPCR value)/(ALU115-qPCR value) ratios in patients with stage I/II and III/IV PAC are significantly higher than in normal volunteers.

In melanoma, the serum ALU247-qPCR values in patients with AJCC stage 0/I, II, III, and IV melanomas are significantly higher than in normal controls. The serum (ALU247-qPCR value)/(ALU115-qPCR value) ratio is elevated with the progression of the disease. While the serum (ALU247-qPCR value)/(ALU115-qPCR value) ratio in stage I is almost the same as in normal controls, it is higher in stages II and III than in normal controls. Patients with stage IV melanomas show significantly higher serum (ALU247-qPCR value)/(ALU115-qPCR value) ratios.

In prostate cancer (PCa), the serum ALU247-qPCR values and the serum LINE1 297-qPCR values in patients with AJCC stage IV PCa are significantly higher than in normal healthy males. The serum uLINE1 values in patients with AJCC stage II-IV PCa are also significantly higher than in normal healthy males.

The invention provides methods for diagnosis, prognosis, and treatment of cancer using free circulating DNA in body fluids as a biomarker. These methods involve determining the integrity of the circulating DNA in a body fluid using the methods described above.

For example, in one embodiment, a diagnostic method of the invention comprises determining the integrity of the circulating DNA in a body fluid sample from a subject suspected of cancer using the methods described above. The integrity of the circulating DNA in the sample is indicative of the presence of the cancer. For instance, if the serum ALU115-qPCR value is higher in a potential breast cancer patient than in a healthy female, the patient is likely to be suffering from breast cancer.

The invention also provides a method for monitoring cancer progression in a subject by monitoring the integrity of the circulating DNA in a body fluid using the methods described above. The change in the integrity of the circulating DNA indicates the progression of the cancer. For example, increasing serum ALU247-qPCR values in a melanoma patient indicate advancement of the cancer. This method can be employed to monitor individuals for disease recurrence after diagnosis and treatment, as well as during treatment to assess tumor regression and response to therapy.

One example of a prognostic method of the invention involves prediction of regional lymph node metastasis positivity, particularly micrometastasis (subclinical disease). The method comprises determining the integrity of the circulating DNA in a body fluid sample from a cancer patient using the methods described above. The integrity of the circulating DNA in the sample is indicative of the possibility of regional lymph node metastasis positivity, particularly micrometastasis. For instance, if the (ALU247-qPCR value)/(ALU115-qPCR value) ratio in a serum taken before surgery from a primary breast cancer patient is higher than that of LVI-negative and/or LN-negative controls, the patient is likely to have regional lymph node metastasis involvement, with micrometastasis.

Further, the invention provides predictive measures of response to cancer therapies. For example, the invention provides a method of predicting the probability of post-operative recurrence of cancer in a subject. The method comprises determining the integrity of the circulating DNA in a body fluid sample from the subject using the methods described above. The integrity of the circulating DNA in the sample is indicative of the possibility of cancer relapse in the subject. For instance, if the serum (ALU247-qPCR value)/(ALU115-qPCR value) ratio in a post-operative breast cancer patient is equivalent to that of patients with stage III or IV primary breast cancer and higher than that of healthy females, the patient is likely to suffer from relapse.

The invention further provides a method of evaluating the efficacy of a cancer therapy. A therapy is administered to a patient suffering from cancer, and the integrity of the circulating DNA in a body fluid sample from the subject is determined using the methods described above. The integrity of the circulating DNA in the sample is indicative of whether the efficacy of the therapy is good or poor. The integrity of the circulating DNA in the sample may be compared with a control value such as a value determined prior to the administration of the therapy to the subject or a value obtained from a healthy subject. Generally, a decreased amount/percentage of circulating DNA released from cancer cells and/or a decreased amount/percentage of unmethylated circulating DNA indicate that the therapy is good, whereas an increased amount/percentage of circulating DNA released from cancer cells and/or an increased amount/percentage of unmethylated circulating DNA indicate that the therapy is bad.

Also with the invention is a method for identifying a compound for treating cancer. The method involves the steps of administering to a subject a test compound and determining the integrity of the circulating DNA in a body fluid sample from the subject using the methods described above. The integrity of the circulating DNA in the sample is indicative of whether the compound is a candidate for treating cancer. The integrity of the circulating DNA in the sample may be compared with a control value such as a value determined prior to the administration of the test compound to the subject or a value obtained from a healthy subject. Generally, if the amount/percentage of circulating DNA released from cancer cells and/or the amount/percentage of unmethylated circulating DNA in the sample is lower than the control value, the test compound is identified as a candidate for treating cancer. Test compounds can be obtained using any of the numerous approaches known in the art. See, e.g., U.S. Pat. No. 6,462,187. Compounds thus identified can be incorporated into pharmaceutical compositions for treating cancer.

In addition, the invention provides a methods for treating cancer. The method involves the steps of identifying a subject suffering from or at risk for developing cancer and administering to the subject an effective amount of a compound of the invention. The term “treating” is defined as administration of a substance to a subject with the purpose to cure, alleviate, relieve, remedy, prevent, or ameliorate a disorder, symptoms of the disorder, a disease state secondary to the disorder, or predisposition toward the disorder. An “effective amount” is an amount of a compound that is capable of producing a medically desirable result in a treated subject. The medically desirable result may be objective (i.e., measurable by some test or marker) or subjective (i.e., subject gives an indication of or feels an effect).

One method provided by the invention for diagnosis, prognosis, and treatment of cancer comprises the steps of obtaining circulating DNA from a body fluid sample, detecting the methylation integrity of the circulating DNA in the sample using a LINE sequence (e.g., LINE1) as a marker, and applying the methylation integrity of the circulating DNA in diagnosis, prognosis, and treatment of cancer as described above.

Markers indicative of the integrity of the circulating DNA in body fluids can be combined in the methods of the invention to achieve optimal function with different degrees of specificity, different degrees of sensitivity, different stages of disease, different ethnic groups or sex, or different geographic distributions. Marker combinations may also be developed to be particularly sensitive to the effect of therapeutic regimens on disease progression. For example, a marker combination of the invention may include any two members or all three members of a group consisting of an ALU sequence integrity marker, a LINE1 sequence integrity marker, and a LINE1 methylation integrity marker. As shown in the examples below, both the combination of ALU, LINE1, and uLINE1 and the combination of ALU247 and uLINE1 exhibit increased specificity compared to each individual marker.

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation. All references cited herein are incorporated by reference in their entirety.

### EXAMPLE I

### Higher Amount of Free Circulating DNA in Serum than in Plasma is Not Mainly Caused by Contaminated Extraneous DNA During Separation

### Introduction

Free circulating DNA has been intensely investigated recently as a biomarker for malignancy and in other diseases (4, 12-18). Free circulating DNA is usually obtained from serum or plasma. The most significant difference between these two sources is the existence of coagulation factors and their related proteins, as well as platelets, in plasma. Several reports indicate that the amount of free circulating DNA is significantly lower in plasma than in serum (19-21), but the reason for this observation is still under controversy (20, 22). If DNA is lost during purification from plasma but not from serum, using serum DNA as a biomarker should be more efficient. However, if extraneous DNA from leukocytes or other sources is accidentally released into serum during its separation from whole blood, using serum DNA would cause erroneous results derived from contaminated DNA. Another possible explanation is unequal distribution of DNA during separation from whole blood; if this is the case, then using serum DNA would increase sensitivity.

To elucidate why the observed amount of DNA is higher in serum than in plasma, we precisely quantified DNA amount in serum and plasma concurrently separated from same blood without possibility of DNA loss. The method we used was quantitative real-time PCR (qPCR) of the ALU repeat, which is the most abundant repeat sequence (1.4×10⁶ copies) in the human genome (23). ALU-qPCR was sensitive enough for using minimally processed serum/plasma as a template without DNA purification.

### Materials and Methods

### Blood Samples and Serum/Plasma Separation

Twenty-four patients with breast cancers (n=12), colorectal cancers (n=8), thyroid cancers (n=2), and thyroid adenomas (n=2) were randomly selected from the clinical database at the John Wayne Cancer Institute (JWCI) in 2005. All patients gave consent for blood sampling according to the guidelines set forth by Saint John's Health Center and JWCI Institutional Review Board (IRB) committee. Ten ml of blood was collected in a CORVAC serum separator tube (Sherwood-Davis & Geck, St. Louis, Mo.) and processed within 6 hrs for serum as follows: the sample was separated by centrifugation (1,000×g, 15 min), and filtered through a 13 mm serum filter (Fisher Scientific, Pittsburgh, Pa.) to remove potentially contaminating cells. An additional 10 ml of blood was collected at the same blood drawn in a sodium citrate tube (Becton Dickinson, Franklin Lakes, N.J.) in a similar manner for plasma isolation.

### Quantification of Free Circulating DNA

To maximize the sensitivity of DNA quantification, ALU repeat that is the most abundant repeat sequence in the human genome was used as a target of qPCR. The primer set was designed to amplify the consensus sequence of ALU and produce an amplicon size of 115 bp. The sequence of the forward primer was 5′-CCTGAGGTCAGGAGTTCGAG-3′ (SEQ ID NO:1); the reverse primer was 5′-CCCGAGTAGCTGGGATTACA-3′ (SEQ ID NO:2).

Human serum and plasma contain many substances that interfere with the PCR reaction, such as proteins which bind to template DNA or DNA polymerase. Therefore, we used minimally preprocessed serum/plasma to eliminate inhibitory factors. We mixed 20 μl of each serum/plasma sample with 20 μl of a preparation buffer that contained 2.5% of Tween-20, 50 mM of Tris, and 1 mM of EDTA. This mixture was digested with 16 μg of proteinase K (Qiagen, Valencia, Calif.) at 50° C. for 40 min, and diluted with 160 μl of Tris-EDTA buffer after 5 min of heat deactivation at 95° C. After centrifugation at 10,000×g for 5 min, 1 μl of supernatant containing 0.1 μl equivalent amount of the serum/plasma was used as a template for ALU-qPCR without purification.

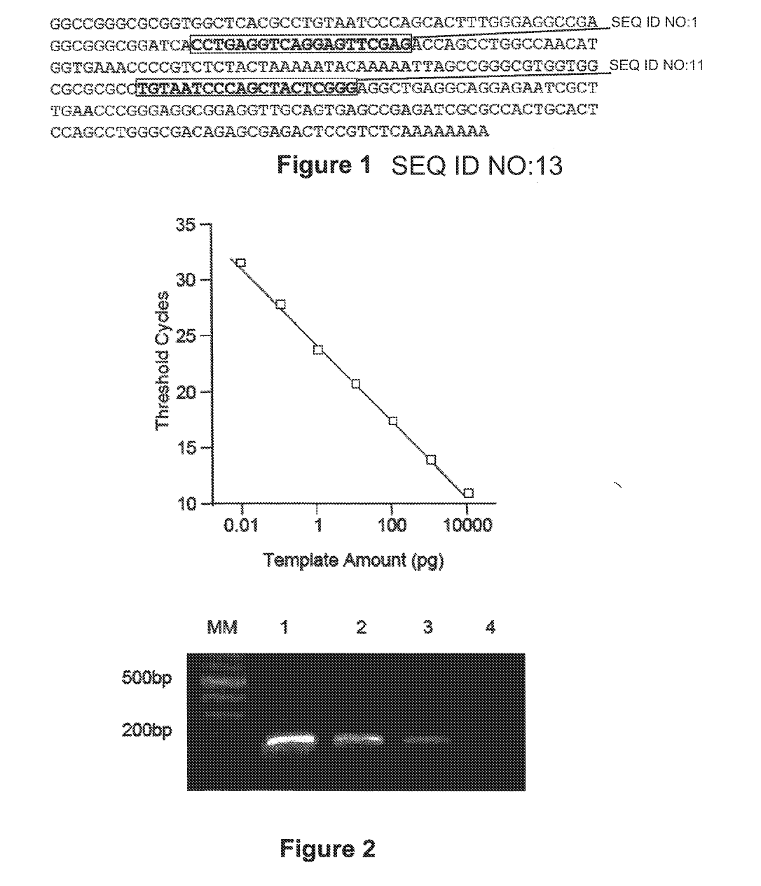

The reaction mixture for each ALU-qPCR consisted of a template, 0.2 μM of forward primer and reverse primer, 1 unit of iTaq DNA polymerase (Bio-Rad Laboratories, Hercules, Calif.), 0.02 μl of fluorescein calibration dye (Bio-Rad Laboratories), and 1×concentration of SYBR Gold (Molecular Probe, Eugene, Oreg.) in a total reaction volume of 20 μl with 5 mM of Mg²⁺. Real-time PCR amplification was performed with a precycling heat activation of DNA polymerase at 95° C. for 10 min, followed by 35 cycles of denaturation at 95° C. for 30 s, annealing at 64° C. for 30 s, and extension at 72° C. for 30 s using iCycler iQ Real-Time PCR Detection System (Bio-Rad Laboratories). The absolute equivalent amount of DNA in each sample was determined by a standard curve with serial dilutions (10 ng to 0.01 pg) of purified DNA obtained from peripheral blood of a healthy volunteer. A negative control (no template) was performed in each reaction plate. PCR products were electrophoresed on 2% agarose gels to confirm product size and specificity of the PCR (FIG. 1A).

To evaluate the effect of inhibitory substances in processed serum/plasma on ALU-qPCR, known amount (10 ng) of purified DNA obtained from PBL of a healthy volunteer was mixed with the reaction mixture of serum/plasma ALU-qPCR of two patients and quantified. The amount of DNA in the template serum/plasma itself was subtracted from the qPCR result.

### Statistical Analysis

The amount of DNA in serum versus plasma was assessed by paired t-test and Deming's regression analysis. The statistical package SAS JMP version 5.0.1 (SAS Institute Inc., Cary, N.C.) and EP Suite 9A version 2.0.a (24) (MarChem Associates, Inc, Concord, Mass.) were used, and P value<0.05 (two-tailed) was defined as significant.

### Results

### Sensitivity and Linearity of ALU-qPCR

ALU-qPCR had linearity ranging from 10 ng to 0.01 pg with a PCR efficiency of 97% and regression coefficient of 0.999 of the standard curve taken from the mean of two sets of serially diluted genomic DNA (FIG. 1B). Thus the lower quantification limit of serum/plasma was 0.1 pg/μl.

### Evaluation of Inhibitory Effect on ALU-qPCR Factors in Serum/Plasma

Triplicate ALU-qPCR results for two sets of 10 ng purified DNA with serum was 10.2±1.8 ng and that with plasma was 9.8±1.2 ng (mean±SD). No significant inhibitory effect by serum or plasma was observed.

### Amount of DNA in Serum and Plasma by ALU-qPCR

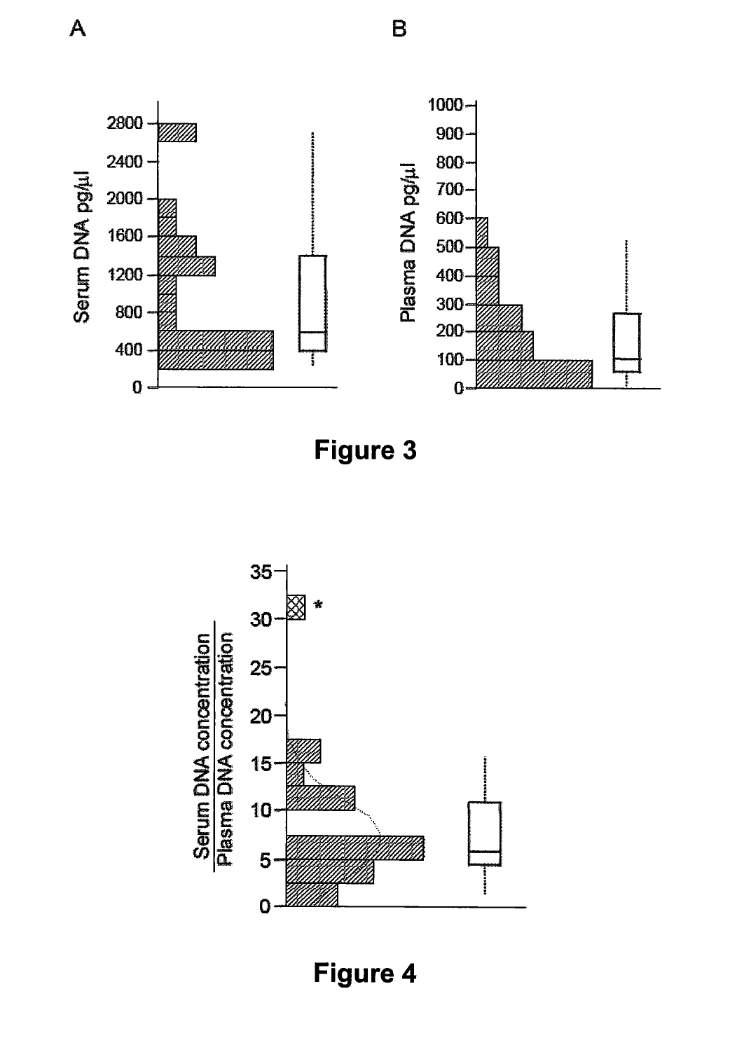

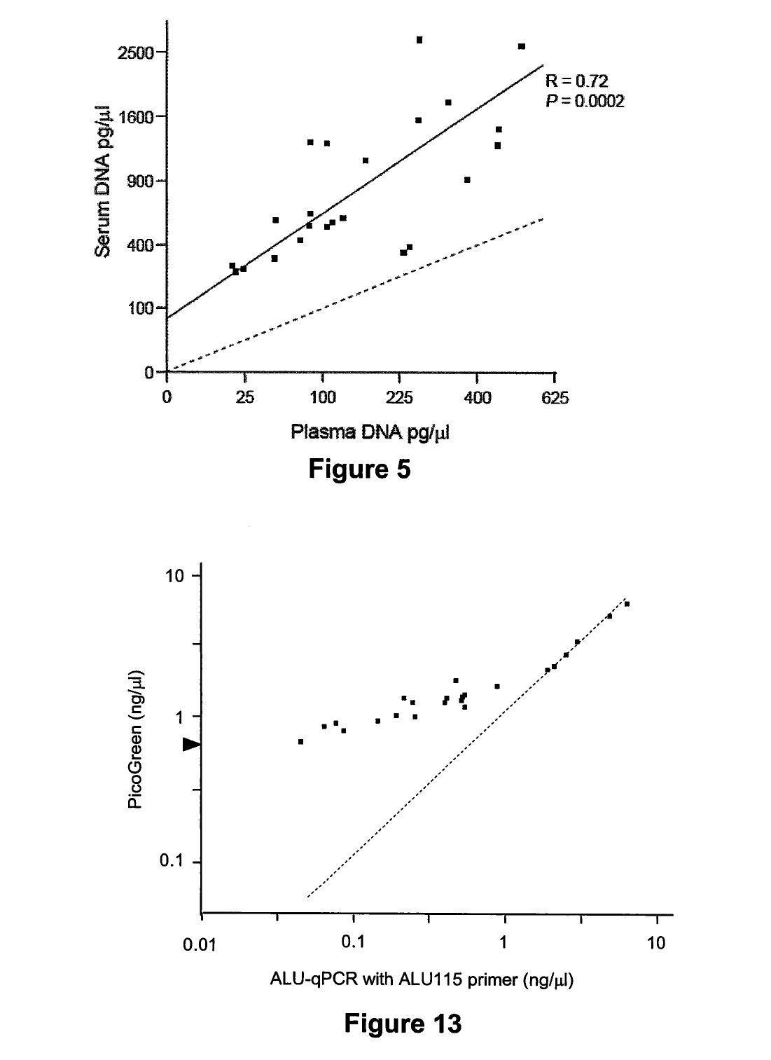

The DNA concentrations of serum and plasma samples by ALU-qPCR were 930±710 pg/μl and 180±150 pg/μl (mean±SD), respectively (FIG. 2). The serum:plasma ratio of DNA in each pair of specimens followed normal distribution (mean: 7.1, SD: 4.2), with the exception of one pair that had a ratio of 32 (FIG. 3). This outlier was therefore deleted from subsequent statistical analysis to avoid overvaluation of serum DNA amount. Serum contained significantly higher amount of DNA than plasma (P<0.0001, paired t-test).

Deming's regression analysis was performed after square-root variable transformation to normalize the distribution of concentrations in serum and plasma (logarithmic transformation was inapplicable because intercept value of serum DNA at a plasma DNA of zero was required). Deming's regression analysis showed positive correlation between DNA amount in serum and that in plasma (R=0.72 and P=0.0002). The intercept value of regression line at a plasma DNA of zero was 8.7 (95% CI: 2.8-14.5) (FIG. 4). Thus the estimated amount of extraneous DNA in serum that was independent to plasma DNA was 76 pg/μl (95% CI: 7.8-210), equivalent to only 8.2% (95% CI: 0.8%-23%) of total amount of serum DNA in average. After subtraction of 76 pg/μl from each qPCR serum value, the serum had 6.1±3.5 (mean±SD) fold greater DNA concentration than the paired plasma in average. This value is concordant with results of previous reports (19,21).

### Discussion

Several reports have demonstrated that free circulating DNA is lower in plasma than in serum (19-21), but none has determined the reason behind this observation. A specious assumption is that leukocytes ruptured during serum separation might release DNA into serum based on the finding that serum DNA concentrations correlated with leukocyte counts (20). This explanation implies that most of DNA in serum is extraneous because serum has about 4- 6 times abundant DNA than plasma (19,21), and thus serum can not be a good biomarker. However, clinical utility of serum has been definitely shown in multiple previous studies (4,12,14,17-18), and we considered that such assumption was incorrect and hypothesized that unequal distribution of DNA during separation from whole blood might be the cause.

Any attempts to investigate the relation between DNA concentrations of serum and plasma have been hindered by the difficulty in handling low amounts of DNA and the complexity of purifying DNA from serum or plasma. Accurate quantification of DNA in small sample volumes of plasma and serum has been very difficult to date. Our preliminary assessment of the sensitivity of PicoGreen (Molecular Probe) assay by a microplate reader, which is a commonly used method for quantification of serum/plasma DNA, showed that the practical lower limit of linear range was around 20 pg/μl in serum/plasma. It was mainly due to the background noise from the photodetector. This assay is not sensitive enough for the accurate assessment of DNA in plasma especially samples from healthy individuals. In addition, because the DNA in plasma or serum is highly truncated (25), recovery of DNA during the purification process is usually not complete and depends on the efficacy of the extraction method. Therefore, we first established a highly sensitive method utilizing ALU repeats for accurate quantification of DNA in serum and plasma without DNA purification. The ALU, which is primate-specific, is the most abundant repeated sequence in the human genome, with a copy number of about 1.4 million per genome (23,26-27). Because most DNA released from apoptotic cells is truncated into a length of 185-200 bp by a cleavage process during apoptosis (25), we designed a primer set for 115 bp amplicon in ALU repeats. This method achieved sufficient sensitivity to accurately quantify with high linearity as little as 0.01 pg of DNA, equivalent to about 1/300 copy of genome in a single cell. As a result, it enabled accurate quantification of free circulating DNA without its purification from serum/plasma because of the low requirement of template DNA. This quantification method has potential clinical applications for measurement of DNA in other body fluids, such as the urine of patients with urinary tract cancers or the saliva of patients with salivary gland cancers.

Because our ALU-qPCR technique eliminated the need for DNA purification, it was proven that the lower level of DNA in plasma was not the result of DNA loss during purification of DNA from plasma. In addition, our results showed a significant positive regression between DNA in serum and plasma; the estimated amount of extraneous DNA in serum was only 8.2% of the total serum DNA. Therefore, it was also proven that the excess amount of DNA in serum is not mainly from extraneous DNA released from leukocytes or other sources during the separation of serum. As a result, the most likely explanation for the difference in serum and plasma DNA levels is unequal distribution of DNA during separation from whole blood. Based on the estimated scale factor of serum DNA in relation to plasma DNA, serum has 6.1-fold more DNA than plasma. This might be the result of electrostatic forces between DNA that is anionic and platelets that are also potently anionic existing only in plasma.

In conclusion, serum contains around six times as much amount of free circulating DNA as plasma. Extraneous DNA such as DNA from cells in blood ruptured during the separation step was minor for explaining the difference between serum and plasma. Most possible explanation was unequal distribution of DNA during separation from whole blood. We advocate that serum is a better specimen source for circulating disease-related DNA as a biomarker.

### EXAMPLE II

### Prediction of Breast Tumor Progression by Integrity of Free Circulating DNA in Serum

### Introduction

Breast cancer is the second leading cause of cancer death among women in the United States, and accounts for one-third of all new cancer cases among women in 2005 (28). The most significant prognostic indicator for patients with breast cancer is axillary lymph node (LN) metastasis (29). Physical examination and diagnostic imaging methods such as ultrasonography or computer tomography are effective for detection of LN metastases when they are relatively large in size. However, there is no clinically established blood test which has the predictive ability to determine regional LN metastasis or distant metastasis. Therefore, the development of a preoperative applicable blood test for detection of LN metastasis of breast cancer is clinically desired.

Tumor-related cell-free DNA circulating in blood is a promising candidate molecular biomarker for malignant tumor detection or prognosis (4,17-18,30). Absolute levels of circulating DNA detected in serum or plasma have been related to presence (31) and prognosis (32) of breast cancer. Methylation of tumor suppressor genes detected in circulating DNA has been demonstrated to have prognostic potential (33-36). Recently, it was shown that the integrity of circulating DNA, measured as the ratio of longer to shorter DNA fragments, is higher in patients with gynecological and breast cancers than in normal individuals (37). Apoptotic cells release DNA fragments that are usually 185-200 bp in length (25); this uniformly truncated DNA is produced by a programmed enzymatic cleavage process during apoptosis (38). In healthy individuals, the main source of free circulating DNA is apoptotic cells. By contrast, DNA released from malignant cells varies in size because pathological cell death in the malignant tumors results not only from apoptosis, but also necrosis, autophagy, or mitotic catastrophe (39). Therefore, elevated levels of long DNA fragments may be a good marker for detection of malignant tumor DNA in blood (40). However, quantification of free circulating DNA in blood has not been practically utilized because of the difficulty in handling the minute amount of circulating DNA.

Recently, we have developed a simple, robust, highly-sensitive, and high-throughput method to directly measure the serum DNA integrity. In this assay, quantitative real-time PCR (qPCR) dependent on DNA fragment size was used to quantify the DNA contained in 0.1 μl of serum. The target of qPCR was the ALU repeated DNA sequence. ALU repeats are the most abundant sequences in the human genome, with a copy number of about 1.4 million per genome (23,26). ALU sequences are short interspersed elements (SINEs), typically 300 nucleotides in length, which account for more than 10% of the human genome (41). In this assay, purification of DNA from serum was not necessary due to a low interference by the serum template. DNA integrity was calculated as a ratio of longer to shorter DNA fragments, quantified by qPCR with two sets of primers (115 bp and 247 bp) for ALU repeats. Applying this approach, we demonstrated that assessing serum DNA integrity was useful for pre-operative prediction of regional LN metastasis in breast cancer. We also demonstrated that serum DNA integrity also directly correlated with advancing American Joint Committee on Cancer (AJCC) breast cancer staging.

### Materials and Methods

### Serum Samples and Clinical and Pathology Information

Serum samples from 51 healthy females and 83 pre-operative females with AJCC stage 0-IV primary breast cancers (8 stage 0, 24 stage I, 27 stage II, 21 stage III, and 3 stage IV) were assessed. Blood was drawn prior to surgery of primary breast cancer. Staging was based on postoperative histopathology findings for stages 0-III, and imaging diagnoses were utilized for stage IV. All patients were selected by the database coordinator from the patients seen between 2000-2005 at the Joyce Eisenberg Keefer Breast Center and the Angeles Clinic at Saint John's Health Center, Santa Monica. All patients in this study gave consent according to guidelines set forth by Saint John's Health Center and JWCI Institutional Review Board.

Serum Preparation for Direct qPCR

Ten ml of blood was collected in a CORVAC serum separator tube (Sherwood-Davis & Geck, St. Louis, Mo.) containing clot activation additive and barrier gel, stored at 4° C., and processed within 6 hrs: the blood was separated by centrifugation (1,000×g, 15 min) and passed through a 13-mm serum filter (Fisher Scientific, Pittsburgh, Pa.) to remove potentially contaminating cells. The serum was immediately cryopreserved at −80° C. To deactivate or eliminate proteins binding to template DNA or DNA polymerase that might invalidate qPCR results, 20 μl of each serum sample was mixed with 20 μl of a preparation buffer that contained 2.5% of Tween-20, 50 mM of Tris, and 1 mM of EDTA. This was digested with 16 μg of proteinase K solution (Qiagen, Valencia, Calif.) at 50° C. for 20 min, followed by 5 min of heat deactivation and insolubilization at 95° C. After subsequent centrifugation of 10,000×g for 5 min, 0.2 μl of supernatant was used as a template for each qPCR reaction.

### Quantitative PCR of ALU Repeats

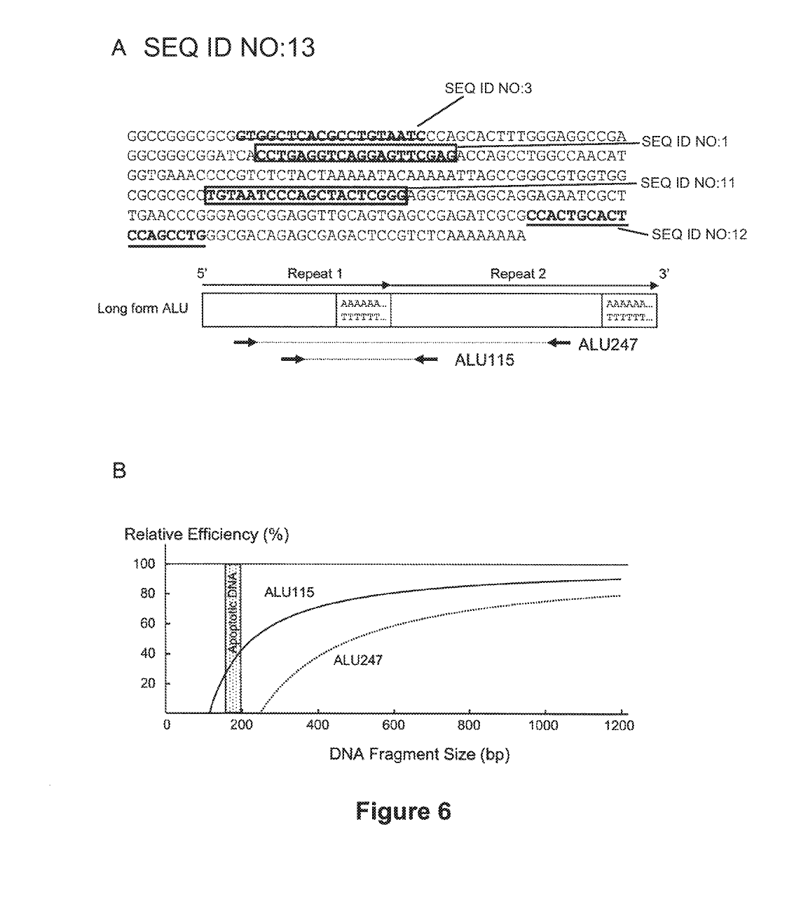

To achieve the highest sensitivity for DNA quantification, we applied a novel qPCR assay that utilizes primer sets to amplify the consensus ALU sequence (FIG. 6A). Two sets of ALU primers were designed: the primer set for the 115 bp amplicon (ALU115) amplifies both shorter (truncated by apoptosis) and longer DNA fragments, whereas the primer set for the 247 bp amplicon (ALU247) amplifies only longer DNA fragments (FIG. 6B). The sequences of the ALU115 primers were forward: 5′-CCTGAGGTCAGGAGTTCGAG-3′ (SEQ ID NO:1) and reverse: 5′-CCCGAGTAGCTGGGATTACA-3′ (SEQ ID NO:2); ALU247 primers were forward: 5′-GTGGCTCACGCCTGTAATC-3′ (SEQ ID NO:3) and reverse: 5′-CAGGCTGGAGTGCAGTGG-3′ (SEQ ID NO:4). ALU115-qPCR results represent the total amount of free serum DNA; the ALU115 primer can amplify most fractions of circulating DNA. ALU247-qPCR results represent amounts of DNA released from non-apoptotic cells, i.e., histopathology classified dead cells. DNA integrity was calculated as the ratio of qPCR results (ALU247-qPCR/ALU115-qPCR). Because the annealing sites of ALU115 are within the ALU247 annealing sites, the qPCR ratio (DNA integrity) is 1.0 when the template DNA is not truncated and 0.0 when all template DNA is completely truncated into fragments smaller than 247 bp.

qPCR reactions were carried out as described above. All qPCR assays were performed in a blinded fashion without knowledge of specimen identity. Mean values were calculated from triplicate reactions.

### Statistical Analysis

Mean values for healthy females and patients with each stage of breast cancer were compared using Dunnett's multiple comparison. Mean values between two groups were compared using Student's t-test. Spearman's ρ coefficients were used to identify the trends between AJCC stage and serum DNA integrity. The effect of clinicopathological characteristics and serum DNA integrity on presence of LN metastasis was assessed using a nominal logistic regression model for multivariate analysis; variables indicated by the univariate analyses (P<0.10) were entered. Linear regression and multiple linear regression was used to assess the possible dependence of mean serum DNA integrity on patient age and tumor clinicopathology information. The statistical package SAS version 5.0.1 (SAS Institute Inc., Cary, N.C.) was used to conduct statistical analyses and MedCalc version 8.0 (MedCalc Software, Mariakerke, Belgium) was used for receiver operating characteristic (ROC) curve analyses. A P value<0.05 (two-tailed) was considered significant.

### Results

### Clinical and Pathology Characteristics of Primary Breast Cancers

The mean age was 45±14 (SD) years for 51 healthy females and 58±12 years for 83 patients with primary breast cancer. Table 1 shows the AJCC stage and histopathology characteristics of breast cancers in patients whose sera were sampled preoperatively. All the stage 0 cancers were ductal carcinomas in situ (DCIS); the mean patient age was 59±11 (SD) years and the mean tumor size was 2.5±1.9 (SD) cm. Of the 42 patients with regional LN metastases, 10 had solitary micrometastases (pN1mi:≦2 mm in size).

TABLE 1
Clinicopathological characteristics of
primary breast cancers (n = 83)

| | Number of | |
|---|---|---|
| Variable | Patients (%) | |
| Sex | | |
| male | 0 | (0) |
| female | 83 | (100) |
| Tumor diameter (cm) (mean ± SD) | 3 | ±2.1 |
| AJCC primary tumor (T) | | |
| Tis | 8 | (10) |
| T1 | 32 | (39) |
| T2 | 28 | (34) |
| T3 | 10 | (12) |
| T4 | 5 | (6) |
| AJCC regional lymph nodes (N) | | |
| N0 | 41 | (49) |
| N1 | 28 | (34) |
| N2 | 10 | (12) |
| N3 | 4 | (5) |
| AJCC distant metastasis (M) | | |
| M0 | 80 | (96) |
| M1 | 3 | (4) |
| AJCC state | | |
| 0 | 8 | (10) |
| I | 24 | (29) |
| II | 27 | (33) |
| III | 21 | (25) |
| IV | 3 | (4) |
| Histopathological grade | | |
| G1 | 23 | (32) |
| G2 | 26 | (37) |
| G3 | 22 | (31) |
| Histopathological type | | |
| DCIS | 8 | (10) |
| invasive ductal | 57 | (69) |
| invasive lobular | 13 | (16) |
| others | 5 | (6) |
| Lymphovascular invasion (LVI)* | | |
| positive | 34 | (41) |
| negative | 48 | (58) |

*LVI in one primary tumor was indefinite.

### Circulating DNA in Serum and AJCC Stage of Primary Breast Cancers

Circulating DNA in serum of patients' blood taken pre-operatively was assessed for levels and integrity of serum DNA. An ALU115-qPCR value represents the absolute total amount of serum DNA. Because the absolute serum DNA levels fitted exponential distribution, we applied logarithmical transformation to each value. The mean logarithmically transformed ALU115-qPCR values in healthy females and patients with stage 0, I, II, III, and IV breast cancer were 2.43±0.10 (SEM), 2.88±0.16, 2.49±0.08, 2.77±0.06, 2.89±0.13, and 3.02±0.11 (log₁₀ of pg/μl), respectively. These mean values were significantly higher in patients with stage II and III cancer than in healthy females (P=0.01 and P<0.0001, respectively). A trend of elevation in stage IV cancer was observed but not significant.

An ALU247-qPCR value represents the absolute amount of longer fragment of serum DNA, supposedly released from non-apoptotic dead cells. The mean logarithmically transformed ALU247-qPCR values in healthy females and patients with stage 0, I, II, III, and IV breast cancer were 1.53±0.09, 2.05±0.20, 1.54±0.11, 2.01±0.08, 2.30±0.15, and 2.55±0.12 (log₁₀ of pg/μl), respectively. These mean values were significantly higher in patients with stage II, III, and IV cancer than in healthy females (P=0.002, P<0.0001, P=0.014, respectively).

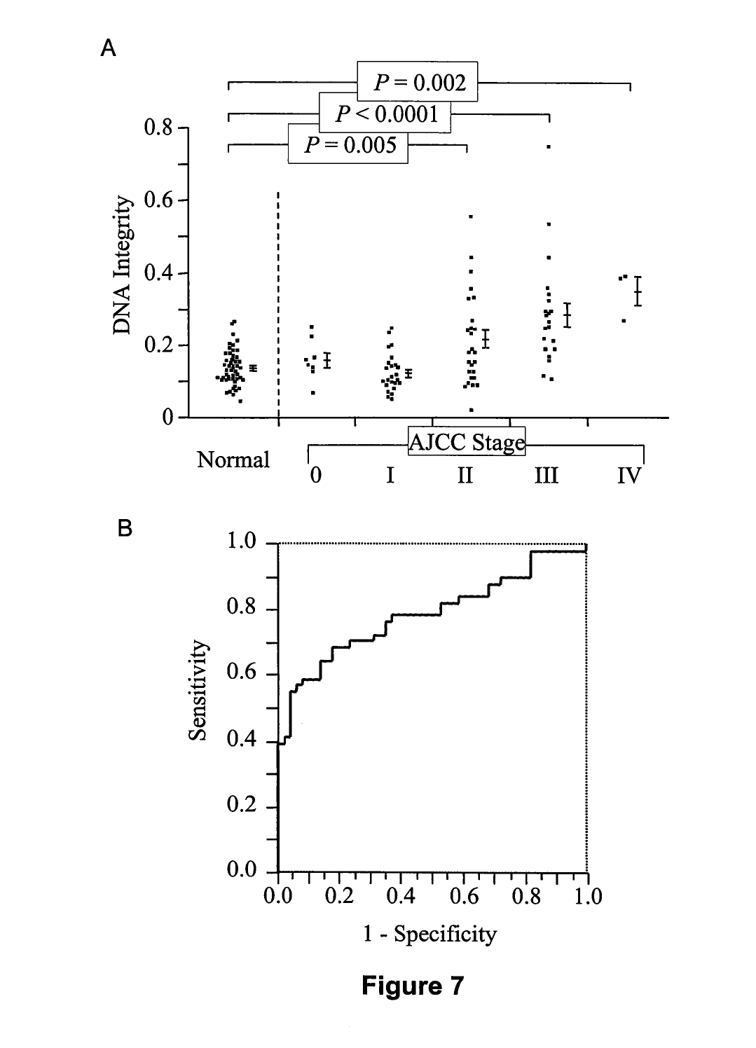

The serum DNA integrity, which represents the ratio of longer DNA fragments in total serum DNA, was calculated as (ALU247-qPCR value)/(ALU115-qPCR value) of each sample. The mean serum DNA integrity in healthy females and patients with stage 0, I, II, III, and IV breast cancer was 0.13±0.01 (SEM), 0.16±0.02, 0.12±0.01, 0.21±0.02, 0.29±0.03, and 0.35±0.04, respectively. The mean values were significantly higher in patients with stage II, III, and IV breast cancer than in healthy females (P=0.005, P<0.0001, and P=0.002, respectively), with a clear discriminative difference (FIG. 7A).

The ALU115- and ALU247-qPCR values showed a trend that increased with AJCC stage. However, serum DNA integrity also showed a more prominent increase with AJCC stage. Spearman's ρ coefficients of AJCC stage with ALU115- and ALU247-qPCR values, and serum DNA integrity were 0.22 (P=0.04), 0.39 (P=0.0003), and 0.54 (P<0.0001), respectively. Because serum DNA integrity had the highest correlation coefficient, it was more representative of tumor progression. The ROC curve of serum DNA integrity for discriminating patients with AJCC stage II-IV breast cancer (N=51) from healthy females had an area under the curve (AUC) value of 0.79 (95% CI: 0.70-0.86) (FIG. 7B). When the specificity was set to 80%, the sensitivity for detection was 69% and the cut-off value of serum DNA integrity was 0.17.

### Serum DNA Integrity and Pathology Characteristics of Primary Breast Cancers

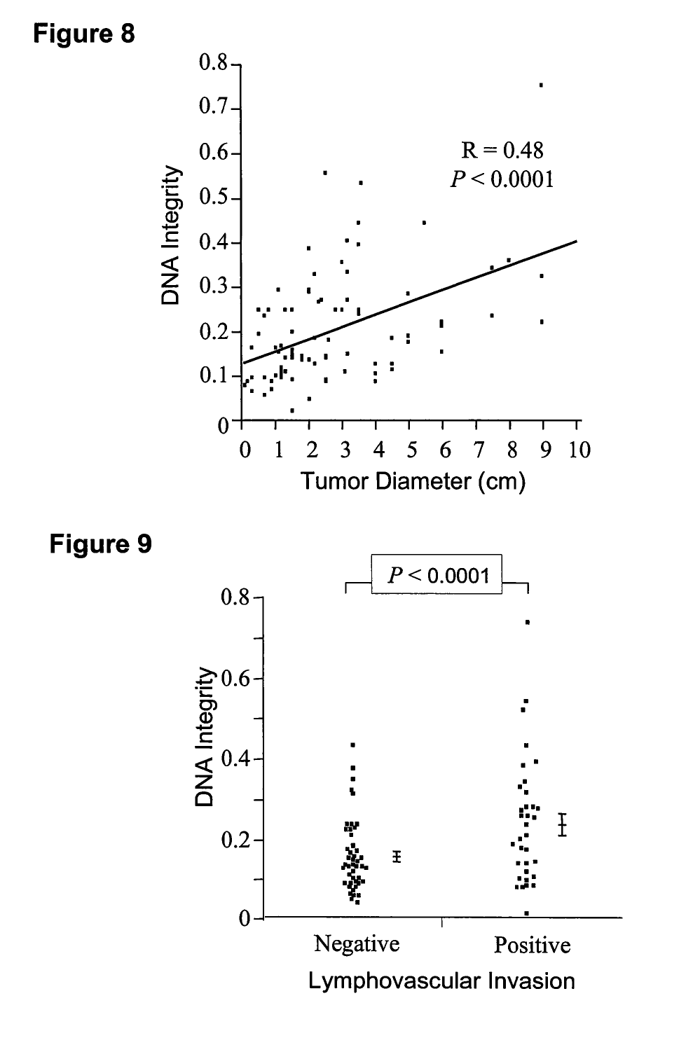

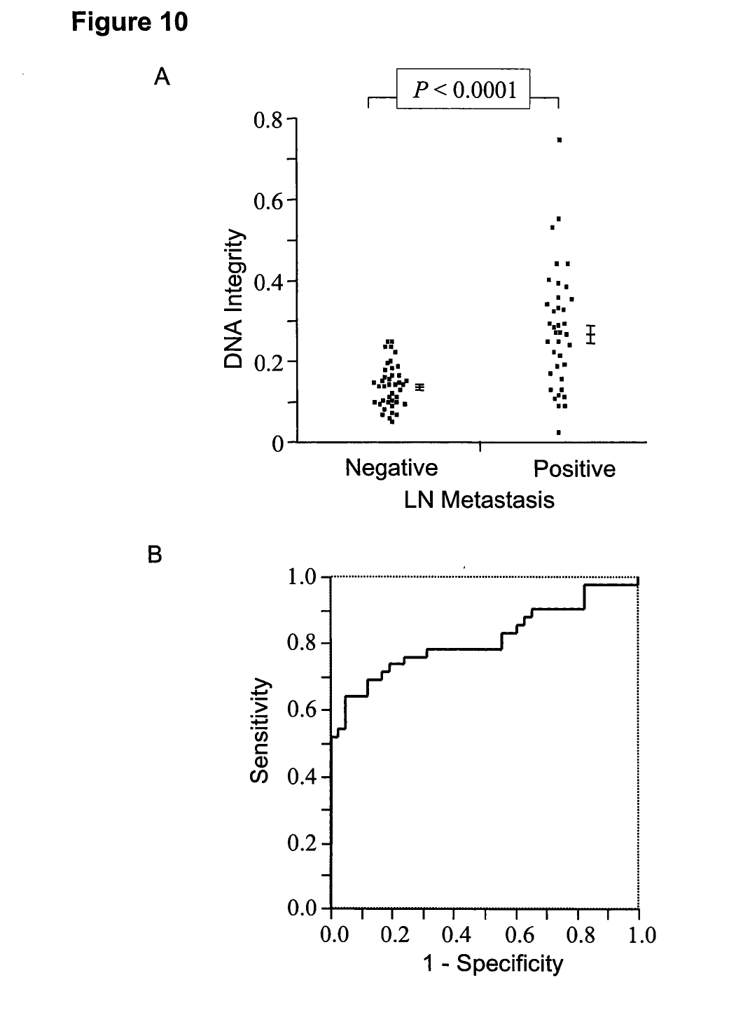

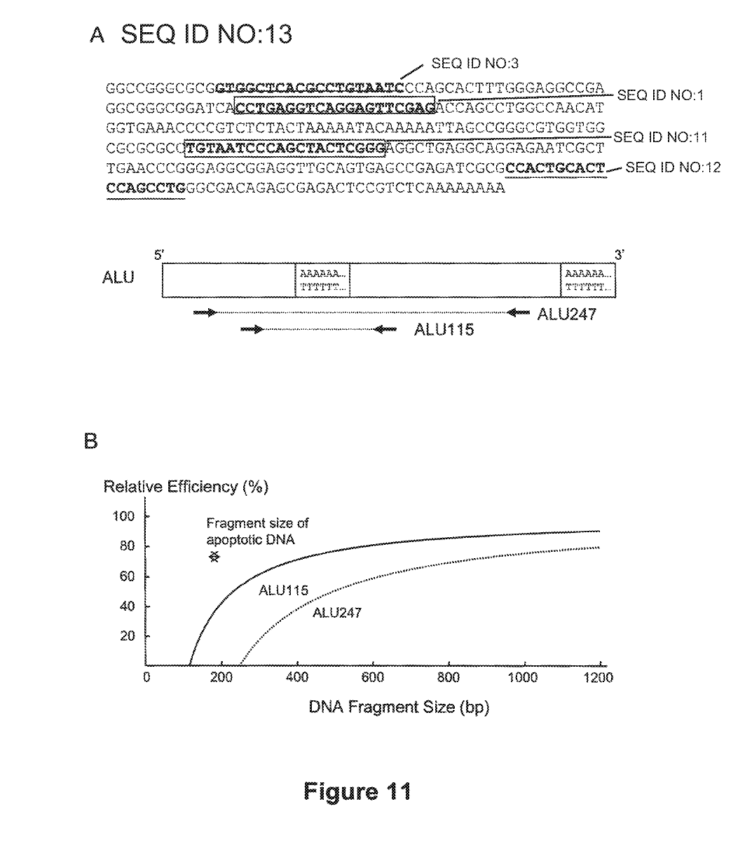

Serum DNA integrity was independent of age in healthy females (P=0.18, univariate regression model) and breast cancer patients (P=0.20, multiple regression model with AJCC stage). Age was not a confounding factor of serum DNA integrity. In the 75 patients with invasive primary breast cancer, serum DNA integrity and size of primary tumor were significantly correlated (R=0.48, P<0.0001) (FIG. 8). In the 82 patients with primary breast cancers whose lymphovascular invasion (LVI) was determined by histopathology, the mean serum DNA integrity in 34 LVT-positive tumors and 48 LVI-negative tumors was 0.25±0.02 (SEM) and 0.17±0.02, respectively. The LVI of one primary tumor was not available. Patients with LVI-positive cancers had significantly higher mean serum DNA integrity than patients with LVI-negative cancers (P<0.0001) (FIG. 9). In 83 patients with primary breast cancer, the mean serum DNA integrity in 42 LN metastasis-positive and 41 LN metastasis-negative cancers were 0.27±0.02 and 0.14±0.02, respectively. Patients with LN metastasis had significantly higher mean serum DNA integrity than patients without LN metastasis (P<0.0001) (FIG. 10A). ROC curve analysis of serum DNA integrity for discriminating patients with regional LN metastasis from patients without LN metastasis had an AUC of 0.81 (95% CI: 0.72-0.89) (FIG. 10B). When the specificity was set to 80%, sensitivity for LN metastasis prediction was 74% and the cut-off value of serum DNA integrity was 0.18.

Multivariate logistic analysis for LN metastasis status with stepwise variable selection from age, tumor size, histological grade, LVI, estrogen receptor, progesterone receptor, HER2 level, and serum DNA integrity was performed. LVI (P<0.0001) and serum DNA integrity (P=0.0002) were significant for LN metastasis. Serum DNA integrity was the only significant pre-operative predictor of LN metastasis.

The 10 patients with micrometastasis (pNlmi) had a mean serum DNA integrity of 0.25±0.02 (SEM), significantly higher than that of LN-negative patients (P<0.0001). This difference suggests that serum DNA integrity may have a clinical utility as a serum biomarker for nodal micrometastasis.

### Discussion

Studies have demonstrated elevated levels of free circulating DNA of various forms in serum or plasma of patients with various types of cancers (16,42-44). Circulating DNA as a molecular biomarker for malignancies can be detected in the form of allelic imbalance, gene methylation, and gene mutation (45). Recently, it has been demonstrated that the length of cell-free plasma DNA in patients with pancreatic cancer was longer than in healthy controls (25). Another study has indicated that the integrity of circulating DNA, calculated from 400 bp and 100 bp qPCR threshold values of a specific gene, may be a molecular biomarker for detection of gynecological and breast cancers (37). It is assumed that the DNA release from malignant tumors into the bloodstream is enhanced by LVI, because direct lymphatic or blood flow through the tumors enables dissemination of viable tumor cells and enhances diffusion of DNA released from dead tumor cells into the bloodstream. As a result, the circulating DNA may be directly related to the tumor progression and the rate of tumor cell turnover representing biological tumor aggressiveness. Therefore, we hypothesized that the integrity of circulating DNA may have a significant prognostic utility for detection of breast cancer progression and LN metastasis.

To measure the integrity of circulating DNA in serum with high sensitivity and reproducibility, we used a newly developed qPCR for ALU repeats. Because the ALU-qPCR assay uses serum directly as the template, it eliminates artifacts associated with DNA purification procedures. Direct qPCR with ALU115 and ALU247 primer sets detected as little as 0.01 pg of DNA (equivalent to about 1/300 of the genome in a single cell) with high linearity. Such high sensitivity minimizes the required volume of template and thus makes the inhibition caused by substances in serum to become negligible (46). The inhibitory effect is further decreased by calculation of DNA integrity as the ratio of two almost identical qPCR assays using same template sera and reagents (except for the primers). During the serum separation, cell lysis due to processing may release long DNA fragments into the serum and raise the DNA integrity. Therefore, we used a filter to remove any remaining contaminant cells from serum. Serum DNA level reportedly rises with spontaneous cell lysis if blood is not processed within 24 hrs (19), but storage of 8 hrs after blood collection at room temperature did not cause significant increase of serum DNA levels with our separation protocol. Therefore, we processed blood within 6 hrs after collection. Our preliminary evaluation of 24 serum samples from patients with various neoplasms in comparison with their paired plasma indicated that only 8.2% of total DNA in serum was extraneous with our protocol (48). In addition, using serum as a template for direct ALU-qPCR was more stable and reproducible than using plasma. Therefore, we used serum as a source for DNA integrity assessment in this study.

The absolute level of serum DNA has been demonstrated to be a potential indicator for cancer existence (31-32). Our results in this study showed that the absolute level of serum DNA measured as ALU115-qPCR value was elevated, on average, in patients with AJCC stage II and III breast cancer. However, we found that the serum DNA integrity had a higher correlation coefficient value with tumor progression than the absolute level of serum DNA. Therefore, serum DNA integrity was considered a better representative for breast cancer progression.

The DNA clearance rate of the patients could directly contribute to the absolute serum DNA level. In contrast, it would not significantly influence the values of DNA integrity because both the amounts of longer and shorter DNA fragments would be similarly affected. In addition, in contrast to absolute DNA levels, which do not reflect how the DNA is released, DNA integrity specifically represents the relative amount of non-apoptotic cell death. Therefore, DNA integrity can be an index of tumor cell death and may be a promising biomarker for tumor detection/progression.

In this study, mean serum DNA integrity was significantly higher in patients with LVI-positive tumors. Furthermore, serum DNA integrity had a highly significant predictive value for LN metastasis. Even in patients with micrometastatic cancers, mean serum DNA integrity showed significantly higher values than healthy females, indicating that this index is elevated with the early stage of LN metastasis. In contrast, mean serum DNA integrity of patients with DCIS was not elevated and was similar to that of healthy females. These findings suggest that serum DNA integrity may be helpful for preoperative evaluation of breast cancer patients with potential regional disease spreading.

To evaluate the utility of serum DNA integrity as a surveillance biomarker for post-operative recurrence, we have additionally assessed 15 females with post-operative recurrence of breast cancer. The mean serum DNA integrity of them was 0.30±0.03 (SEM), and it was equivalent to that of the patients with stage III or IV primary breast cancer, and higher than the corresponding mean value in healthy females. This preliminary finding indicated possible utility of serum DNA integrity for surveillance of postoperative recurrence.

In summary, mean serum DNA integrity was higher in sera drawn from patients with advancing stages of breast cancer than in sera drawn from healthy females. Evaluation of serum DNA integrity achieved 69% sensitivity for detection of AJCC stage II-IV primary breast cancer with a specificity of 80%, and sensitivity of 74% for LN metastasis of primary breast cancer with a specificity of 80%. This rapid and minimally invasive approach to index tumor-related circulating DNA has potential as a screening tool and for the preoperative prediction of LN metastasis.

### EXAMPLE III

### Elevated Integrity of Free Circulating DNA in Sera of Colorectal and Periampullary Cancer Patients: A Direct Quantitative PCR for ALU Repeats

### Introduction

Colorectal cancer (CRC) and periampullary cancers (PAC; primarily pancreatic cancer) were the third and fourth leading causes of cancer-related deaths between 1995-2000 in the United States, respectively (28). Furthermore, the cancer death-rate of advanced CRC remains unsatisfactory and the mortality from pancreatic cancer is among the worst of all cancers. About 80% of patients with pancreatic cancer present with unresectable disease, resulting in a five-year relative survival rate of only 4% (28). The key for improvement of prognosis and treatment is early diagnosis of malignancy. However, most CRC and PAC are asymptomatic and rarely produce symptoms in early stage disease. Screening with established tumor markers for gastrointestinal cancers, such as carcinoembryonic antigen (CEA) or CA19-9, has limited efficiency because of erratic detection and elevation in benign disorders (49-50). Therefore, a development of a widely applicable sensitive screening tool is clinically desired.

Free circulating DNA in serum or plasma has been proposed as a diagnostic and prognostic biomarker for malignant tumors (4,17-18). Recently, it was reported that elevation of DNA integrity in plasma, measured by a relative increase in longer versus shorter DNA fragments, can predict the existence of gynecological and breast cancers (37). In the report, plasma DNA integrity values were derived from threshold cycle numbers assessed by real-time quantitative PCR (qPCR) for two amplicons (400 bp and 100 bp) of a specific gene. The premise is that DNA released from necrotic malignant cells varies in size, whereas DNA released from apoptotic cells is uniformly truncated into 185-200 bp (25). Because the main source of free circulating DNA in healthy individuals is apoptotic cells, a preponderance of longer fragment DNA can be a marker for malignant tumor detection (40). However, the integrity of free circulating DNA is not yet practical for clinical use because its sensitivity and specificity have not been validated. A potential limitation may be the purification of DNA from serum or plasma, which decreases DNA yield. DNA loss may be inversely dependent on fragment size, which would affect DNA integrity values.

Recently, we developed a simple, robust, highly-sensitive, and high-throughput method to measure the integrity of free circulating DNA in serum. It utilizes quantitative real-time PCR (qPCR) for ALU repeats on 0.1 μl equivalent volume of serum as a template without DNA purification. The ALU is the most abundant repeated sequence in the human genome, with a copy number of about 1.4 million per genome (23,26). ALU sequences are short interspersed elements (SINEs), typically 300 nucleotides in length, which account for more than 10% of the human genome (41). ALU elements multiply within the genome in a retroposition process through RNA polymerase III-derived transcripts from evolution (27,51). Therefore, qPCR of ALU repeats with an properly-designed primer set can dramatically increase the sensitivity of size-dependent DNA measurement. In this pilot study of CRC and PAC, we describe this method in detail and validate the practical utility of serum DNA integrity as a sensitive tumor biomarker.

### Materials and Methods

### Serum Samples and Clinicopathological Information

Serum samples from 32 patients with CRC, 19 patients with PAC, and 51 healthy volunteers were assessed. PAC consisted of 15 pancreatic ductal adenocarcinomas, 2 ampullary cancers, 1 acinar cancer, and 1 duodenal cancer. Blood was drawn prior to therapeutic intervention. Patients were selected by the database coordinator based on those patients treated between 1997 and 2005 at JWCI and at UCLA. All patients in this study were consented according to the guidelines set forth by JWCI and UCLA Institutional Review Board (IRB) committees. Of 32 patients with CRC, 3 had American Joint Committee on Cancer (AJCC) stage I disease, 14 had stage II, 6 had stage III, and 9 had stage IV. Of 19 patients with PAC, 2 had AJCC stage I disease, 9 had stage II, 1 had stage III, and 7 had stage IV. Staging was based on postoperative pathological findings for resected cancers or diagnostic imaging for unresectable cancers. Clinicopathological data were obtained after IRB approval for all patients.

### Quantitative PCR of ALU Repeats (ALU-qPCR)

The target for ALU-qPCR in this study was a consensus sequence of human ALU interspersed repeats (Figure IIA). Two sets of primers for ALU115 and ALU247, as described above, were used in the ALU-qPCR reactions.

qPCR reactions were carried out as described above. All qPCR assays were performed in a blinded fashion without knowledge of the specimen identity, and the mean values were calculated from triplicate reactions. PCR products were electrophoresed on 2% agarose gels to confirm product size and specificity of the PCR.

DNA integrity was calculated as the ratio of qPCR results with the two primer sets: Q₂₄₇/Q_{115,} where Q₁₁₅ and Q₂₄₇ are the ALU-qPCR results with ALU115 and ALU247 primers, respectively.

### Serum Preparation and Direct ALU-qPCR

Serum preparation was carried out as described above.

### Evaluation of ALU-qPCR

Because the ALU-qPCR method used in this study was newly developed, we initially evaluated the performance of ALU-qPCR itself using purified DNA or serum as a template.

The sensitivity and linearity of ALU-qPCR with ALU115 or ALU247 primers was evaluated using a serially diluted, known amount of purified DNA obtained from peripheral blood leukocytes (PBL) of a healthy volunteer. In addition, the results of ALU-qPCR with ALU115 primer for serum DNA were compared with the results of the PicoGreen (Molecular Probes) reagent which is a sensitive fluorescent nucleic acid stain for quantifying double-stranded DNA. Serum DNA of 15 normal volunteers and 8 patients with PAC (evaluation set) was extracted and purified by a conventional technique; 500 μl of each separated and filtered serum was digested with 400 μg of proteinase K solution (Qiagen) along with 1% SDS, and DNA was purified by phenol-chloroform-isoamylalcohol and ethanol precipitation as previously described (18).

The reproducibility of direct ALU-qPCR with ALU115 and ALU247 primers was assessed by triplicate reactions using 0.1 μl equivalent volume of each serum of evaluation set.

The interfering effect of substances in serum on direct ALU-qPCR was also evaluated. Samples containing 10 ng of purified PBL DNA (P), 0.1 μl equivalent volume of serum (S), and a mixture of them (P+S) were prepared for each serum of evaluation set. DNA amounts in (P), (S), and (P+S) were separately quantified by ALU-qPCR with ALU115 and ALU247 primers. The interfering effect of serum on ALU-qPCR was calculated as follows: 1.0-(Q_{115(P+S)}−Q_{115(S)} )/Q_{115 (P)} for ALU115 primers and 1.0−(Q_{247(P+S)}−Q_{247(S)}))/Q_{247(P)} for ALU247 primers where Q₁₁₅₍ₓ₎ and Q₂₄₇₍ₓ₎ are the ALU-qPCR results on sample × with ALU115 and ALU247 primers, respectively. The interfering effect of serum on DNA integrity was calculated as follows: 1.0-(Q_{247(P+S)}−Q_{247(S)})/(Q_{115(P+S)}−Q_{115(S)})

### Statistical Analysis

The comparison of absolute level or integrity of serum DNA and clinicopathological characteristics was assessed using Dunnet's multiple comparison. Receiver operating characteristic (ROC) curve analysis was used to assess the discriminating ability of assessments. The statistical package SAS JMP version 5.1 (SAS Institute Inc., Gary, N.C.) was used to conduct statistical analyses. A P value<0.05 (two-tailed) was considered as significant.

### Results

### Sensitivity, Linearity, and Reproducibility of ALU-qPCR

To evaluate the performance of ALU-qPCR as a length-dependent quantification of DNA, we have tested sensitivity, linearity, and reproducibility of ALU-qPCR using purified genomic DNA.

Calculated relative efficiency of ALU-qPCR in relation to fragment length of template DNA was shown in Figure HB. The solid line shows estimated efficiency of ALU-qPCR with ALU115 primer set: 0% in <115 bp and 90% at 1150 bp DNA fragments; the dotted line is for ALU247 primer set: 0% in <247 bp and 90% at 2470 bp. As a result, DNA fragments between 115 bp to 247 bp, which covers the length of DNA cleaved by apoptotic process, can be amplified with ALU115 primers but not with ALU247 primers.

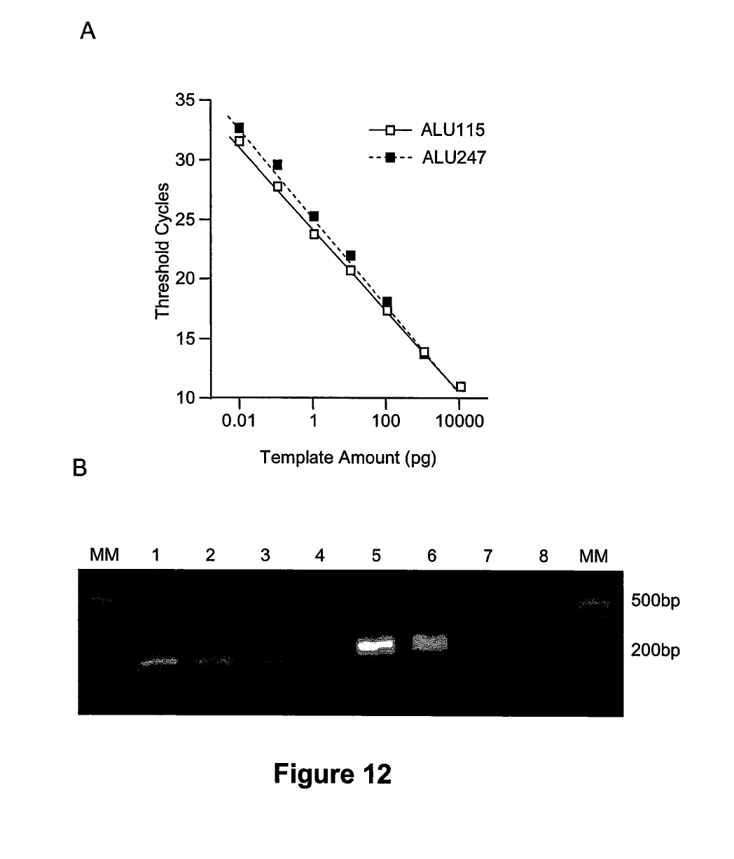

Threshold cycles of ALU-qPCR with ALU115 or ALU 247 primers on serially diluted genomic DNA (10 ng to 0.01 pg) obtained from PBL of a healthy volunteer are shown in FIG. 12A. In both primer sets, linearity was maintained in 10⁶ range and logarithmic regression lines had R=0.998 for ALU115 and R=0.999 for ALU247 primers; sensitivity was as low as 0.01 pg, equivalent to about 1/300 copy of genome in a single cell. Agarose gel views by electrophoresis of PCR products of ALU115 and ALU247 primer sets on 10, 1, 0.1, and 0.01 pg of genomic DNA templates at 28 cycles of thermal cycling confirmed, that the target sequence was specifically amplified without major aberrant bands (FIG. 12B).

The sensitivity of ALU-qPCR was also evaluated using clinical samples from 15 normal volunteers and 8 patients with PAC (FIG. 13). The amount of DNA purified from 500 μl of each serum specimen in the evaluation set was measured in two ways: by the PicoGreen assay consuming 1/10 of total extracted DNA; and by ALU-qPCR with ALU115 primers consuming only 1/5000 of total extracted DNA. Most of the serum samples from healthy volunteers contained too low amount of DNA for accurate quantification by the PicoGreen assay. In contrast, ALU-qPCR had enough sensitivity and linearity for serum DNA quantification. ALU-qPCR results of specimens having relatively high serum DNA levels showed 1:1 linearity with the PicoGreen assay (FIG. 13).

The reproducibility of direct ALU-qPCR was evaluated by triplicate reactions using 0.1 μl equivalent volume of each serum specimen in the evaluation set. The median replicate coefficient of variation (CV) values for ALU115 and ALU247 primers were only 0.09 (interquartile range (IQR): 0.07-0.14) and 0.17 (IQR: 0.08-0.24), respectively. These CV values were equivalent to those of qPCR with specific primers for other genes.

### Interfering Effect of Serum on Direct ALU-qPCR

Because unpurified serum DNA directly used as a template for ALU-qPCR can inhibit the reaction efficiency, we have tested its interfering effect on ALU-qPCR.

The median interfering effects of serum on direct ALU-qPCR with ALU115 and ALU247 primers were 0.09 (IQR: 0.03-0.20) and 0.24 (IQR: 0.09-0.37), respectively. The median interfering effect on DNA integrity was 0.12 (IQR: 0.08-0.18). Because the high sensitivity of ALU-qPCR lowered the requirement of serum template for ALU-qPCR, the interfering effect of serum was limited.

### Absolute Level and Integrity of Serum DNA

Normal volunteers: Mean age of 51 normal volunteers consisted of 18 males and 33 females was 48±11 (SD) years. Mean absolute serum DNA level in normal volunteers was 0.34±0.25 (SEM) ng/μl. Mean serum DNA integrity was 0.13±0.01. Absolute level and integrity of serum DNA in normal volunteers was independent of sex and age.

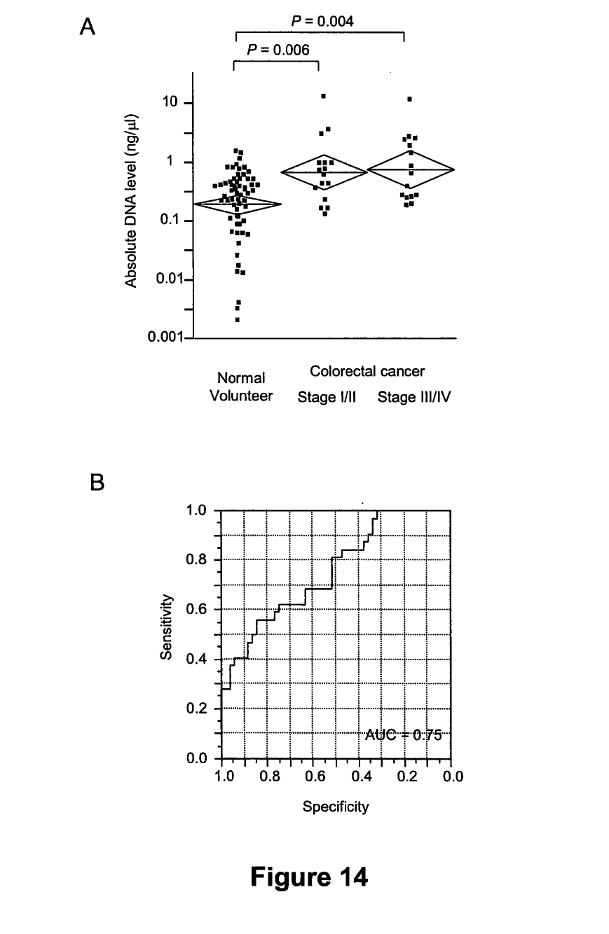

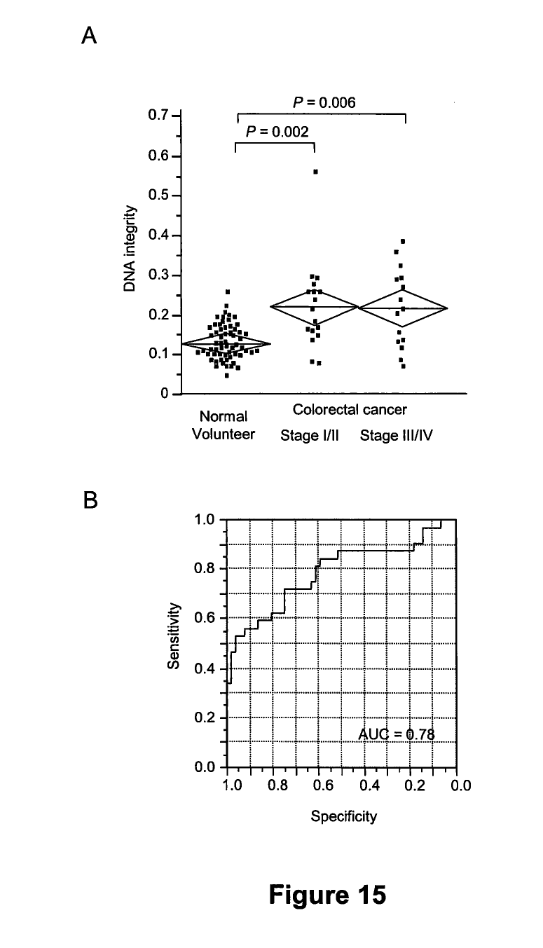

Patients with CRC: Mean age of 32 patients with CRC consisted of 19 males and 12 females was 66±14 (SD) years. Mean absolute serum DNA levels in patients with stage I/II and stage III/IV CRC were 1.63±0.43 and 1.73±0.45 ng/μl, respectively, significantly higher than in normal volunteers (P=0.006 and P=0.004, respectively) (FIG. 14A). ROC curve for discriminating patients with CRC from healthy volunteers by absolute DNA levels had an area under curve (AUC) of 0.75 (FIG. 14B). Mean serum DNA integrity in patients with stage I/II and stage III/IV CRC was 0.22±0.02 and 0.22±0.02, respectively, significantly higher than in normal volunteers (P=0.002 and P=0.006, respectively) (FIG. 15A). ROC curve for discriminating patients with CRC from normal volunteers by serum DNA integrity had an AUC value of 0.78 (FIG. 15B). ROC curve for serum DNA integrity and absolute serum DNA level were similar, indicating that serum DNA integrity may be equivalent to absolute serum DNA level with respect to CRC detection.

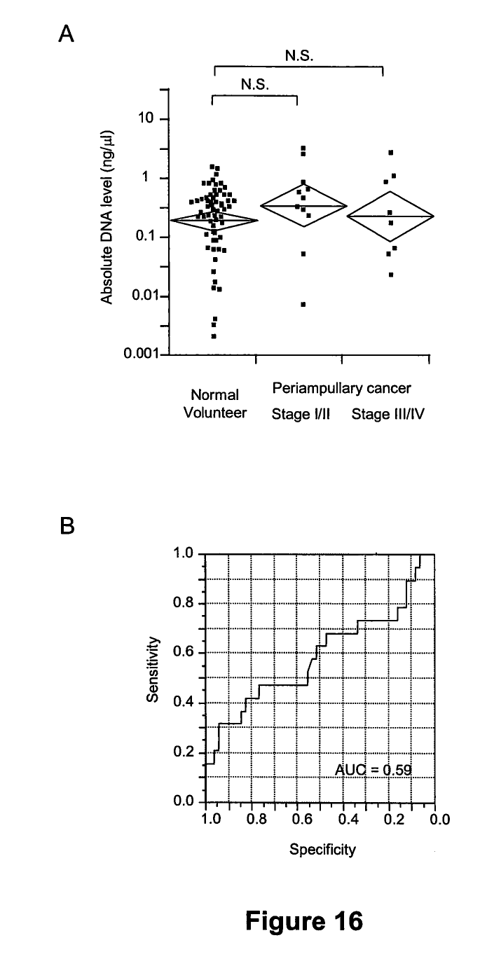

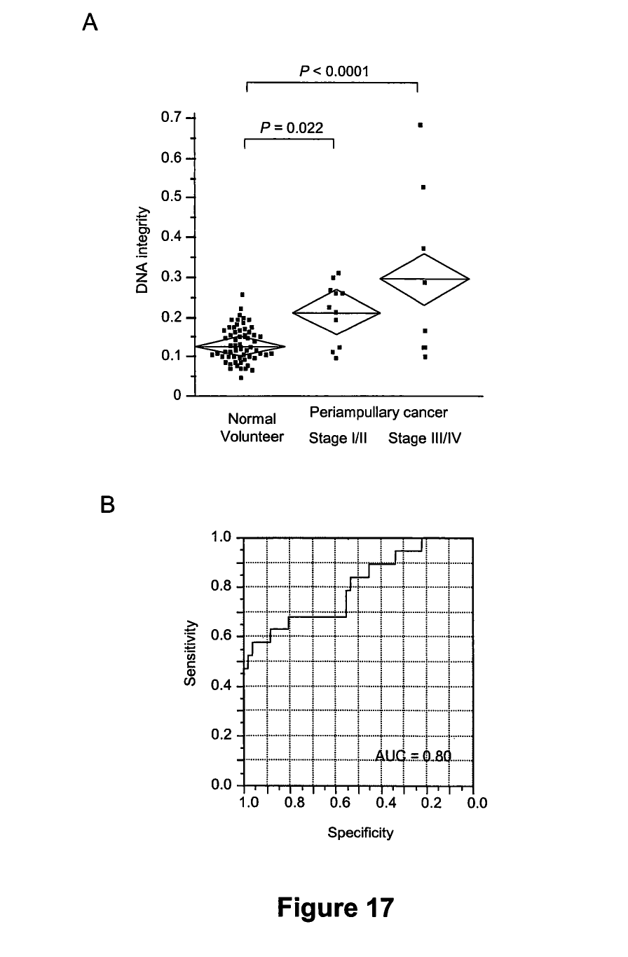

Patients with PAC: Mean age of 19 patients with PAC consisted of 12 males and 7 females was 68±9 (SD) years. Mean absolute serum DNA levels in patients with stage I/II and stage III/IV PAC were 0.84±0.53 and 0.66±0.62 ng/μl, respectively. There was no significant difference between cancer patients and normal volunteers (P=0.85 and P=0.98, respectively) (FIG. 16A). ROC curve for discriminating patients with PAC from healthy volunteers by absolute DNA levels had an AUC of only 0.59 (FIG. 16B). Mean serum DNA integrity in patients with stage I/II and stage III/IV PAC was 0.22±0.03 and 0.30+0.03, respectively, significantly higher than in normal volunteers (P=0.022 and P<0.0001, respectively) (FIG. 17A). ROC curve for discriminating patients with PAC from normal volunteers by serum DNA integrity had an AUC of 0.80 (FIG. 17B). This was greater than AUC for absolute serum DNA level, indicating that serum DNA integrity was more informative than absolute serum DNA level with respect to detection of PAC.

### Discussion

Free circulating DNA in serum/plasma is a promising biomarker of cancer because it contains DNA released from dead tumor cells. Detection of cancer-specific somatic mutations of genes such as K-ras has been demonstrated in plasma/serum of patients with CRC or pancreatic cancer (6,52-53). Cancer detection by quantifying the absolute level of free circulating DNA in serum/plasma has been also reported in multiple publications (16,42-44). Plasma DNA integrity was reported to be a predictor of gynecological and breast cancer existence (37).

Among the aforementioned indices, we considered that DNA integrity may represent cancer cell death and thus could be a widely applicable biomarker for cancer existence or progression. However, difficulty in handling the very low levels of DNA in serum/plasma has been a technical barrier for practical applications. DNA purification steps introduce loss of DNA which in itself is a problem in the assessment of free circulating DNA. In addition, since the recovery rate of serum/plasma DNA depends on DNA fragment size, it becomes a critical fluctuating factor for DNA integrity. To overcome these problems, we developed the ALU-qPCR method to directly measure the absolute amount and integrity of DNA in serum.

Because the ALU is the most abundant repeated sequence (23,26), ALU-qPCR has enough high sensitivity for direct assessment of serum. Elimination of DNA purification in direct ALU-qPCR stabilized the ratio of shorter and longer DNA fragments in serum. In addition, a standard curve created by simultaneously performed qPCR on serially diluted genomic DNA in each reaction plate minimized the variance of ALU-qPCR results between reaction plates. Elimination of DNA purification also reduced the reagent and labor costs for the assessment, which is an important factor for implementation of screening tools. For a large-scale assessment in future, direct ALU-qPCR is easily applicable for robotic automations. The extremely small volume of serum needed for this assessment is compatible with its use as a screening tool.

The levels of free circulating DNA are 4-6 fold higher in serum than plasma (19,21,48). Because this difference does not reflect contaminated extraneous DNA during separation, serum is a better specimen source for circulating disease-related DNA (48). However, during serum separation, cell lysis of PBL may cause an artificial elevation of DNA integrity. Elevation of serum DNA was reportedly observed with overnight clotting after blood drawing (19). Therefore, we processed blood within 6 hrs after blood drawing.

As demonstrated in this pilot study, serum DNA integrity was a clinically useful biomarker for detecting CRC and PAC as demonstrated with the ROC curves. Serum DNA integrity was significantly increased even in localized CRC and PAC. Therefore, it may be useful for mass screening of malignant diseases. However, because any necrotic or mechanically ruptured cells release longer DNA fragments, patients with non-neoplastic diseases such as injury (54), acute inflammation or infarctions may have high serum/plasma DNA integrity. Pregnancy may cause a false positive because of fetal DNA in maternal bloodstream (55). Such conditions may represent exclusion criteria for the assay as a screening tool for malignancy.

Absolute level of serum DNA had a predictive value for CRC but not for PAC in this pilot study. Therefore, we consider serum DNA integrity to be a better biomarker than absolute serum DNA level. However, sera from certain cases with advanced cancers in this study showed very high ALU-qPCR values with ALU115 primers which resulted in lower serum DNA integrity. In such cases, absolute serum DNA may be a better serum biomarker than DNA integrity. Therefore, a combined index of absolute level and integrity of serum DNA may decrease false negatives for cancer detection.

Colorectal cancer is the third leading cause of cancer-related deaths in the US (28). Methods to decrease mortality and increase survival have resulted from implementation of screening programs. Frequently, patients require colonoscopy, an invasive and relatively expensive examination, to provide screening and diagnostic measures (56). Subsequent early operative intervention has resulted in improved survival (57). Despite these measures, a disproportionately high percentage of patients (15-20%) with early stage CRC suffer from local and distant recurrences (28). Surveillance mandates use of CEA, colonoscopy, and radiographic imaging. Such follow-up is expensive and not cost-effective for the vast majority of postoperative patients (58). DNA integrity has potential to help in screening and surveillance.

Periampullary cancers, specifically pancreatic cancer, represent the fourth most common cause of cancer-related deaths in the US (28). Of the estimated 32,190 new cases of pancreatic cancer in 2005, 80%-90% of patients will present with clinically apparent metastatic disease or radiographic evidence of unresectability (28). Unfortunately, effective screening programs for pancreatic cancer have not been developed and patients are often detected incidentally or after development of symptoms. DNA integrity, as an easy, simple, cheap screening method, could provide the impetus to initiate more aggressive radiographic evaluation. For those who are fortunate to have early diagnosis, surgical resection offers the only hope for cure. Despite adherence to rigorous surgical technique and histopathology diagnosis, up to 80% of patients will suffer early, locoregional recurrences (59-60). Therefore, DNA integrity can also be used as a postoperative surveillance tool.

In conclusion, direct ALU-qPCR is a simple, robust, highly-sensitive, and high-throughput method for clinical use to measure the integrity of free circulating DNA in serum. Elimination of DNA purification steps reduced technical artifacts and the reagent and labor cost. Serum DNA integrity was significantly elevated in patients with CRC and PAC. Serum DNA integrity is a promising biomarker for detecting CRC and PAC. The high sensitivity of direct ALU-qPCR suggests that it may be applicable for measurement of DNA level or DNA integrity in other human body fluids.

### EXAMPLE IV

### Elevated DNA Integrity in Circulating DNA of Stage IV Melanoma Patients

### Materials and Methods

### Serum Samples and Clinicopathological Information

We assessed free circulating DNA levels in serum separated from peripheral blood of 65 healthy volunteers and 83 AJCC stage 0 to IV melanoma patients. Institutional Review Board approval and histopathological confirmation from Saint John's Health Center and John Wayne Cancer Institute joint committee were obtained before the initiation of this study.

Serum Preparation for Direct qPCR

Serum was separated from 10 ml of blood collected in a CORVAC serum separator tube (Sherwood-Davis & Geck, St. Louis, Mo.) and processed within 2-6 hrs after blood drawing as described above.

qPCR of ALU Repeat

Two primer sets were used for qPCR of ALU115 and ALU247 as described above. DNA integrity was calculated as (qPCR result with ALU247 primers)/(qPCR result with ALU115 primers).

Reaction of qPCR was performed on the iCycler iQ Real-Time thermocycler (Bio-Rad Laboratories, Hercules, Calif.). For each direct qPCR, the reaction mixture consisted of 0.1 μl equivalent amount of processed serum, 0.2 μM of forward primer and reverse primer, 1 u of iTaq DNA polymerase (Bio-Rad Laboratories), 0.01 μl of Fluorescein Calibration Dye (Bio-Rad Laboratories), and 1× concentration of SYBR Gold (Molecular Probe, Eugene, Oreg.). For the evaluation set, extracted DNA from 0.25 μl of serum was used as the template instead. Real-time PCR amplification was performed in a 20 μl reaction volume with 5 mM of Mg²⁺ for 35 cycles: 30 s at 95° C., 30 s at 64° C., and 30 s at 72° C. after activation of DNA polymerase at 95° C. for 10 min. Absolute equivalent amount of DNA in each sample was determined using a standard curve with serial dilutions (10 ng to 0.01 pg) of genomic DNA of PBL obtained from a healthy volunteer. A negative control without template was performed in each reaction plate. All samples were analyzed in blind fashion without prior knowledge of the specimen identity. All qPCR reactions were triplicated and the mean values were calculated and used for subsequent analysis.

### Statistical Analysis

The relation between DNA integrity and clinicopathological characteristics was assessed using Wilcoxon's rank sum test for univariate analysis and logistic regression model for multivariate analysis. Dunnett's multiple comparison was used for comparisons between normal control and other groups. Spearman's p coefficients were used to detect the trends between AJCC stages and qPCR results or DNA integrity. Variables suggested by the univariate analyses (P<0.10) were entered into the multivariate analyses. The statistical package SAS JMP version 5.0.1 (SAS Institute Inc., Cary, N.C.) was used to conduct statistical analyses. A P value <0.05 (two-tailed) was considered as significant.

### Results

### Amount of Circulating Long/Short DNA in Serum

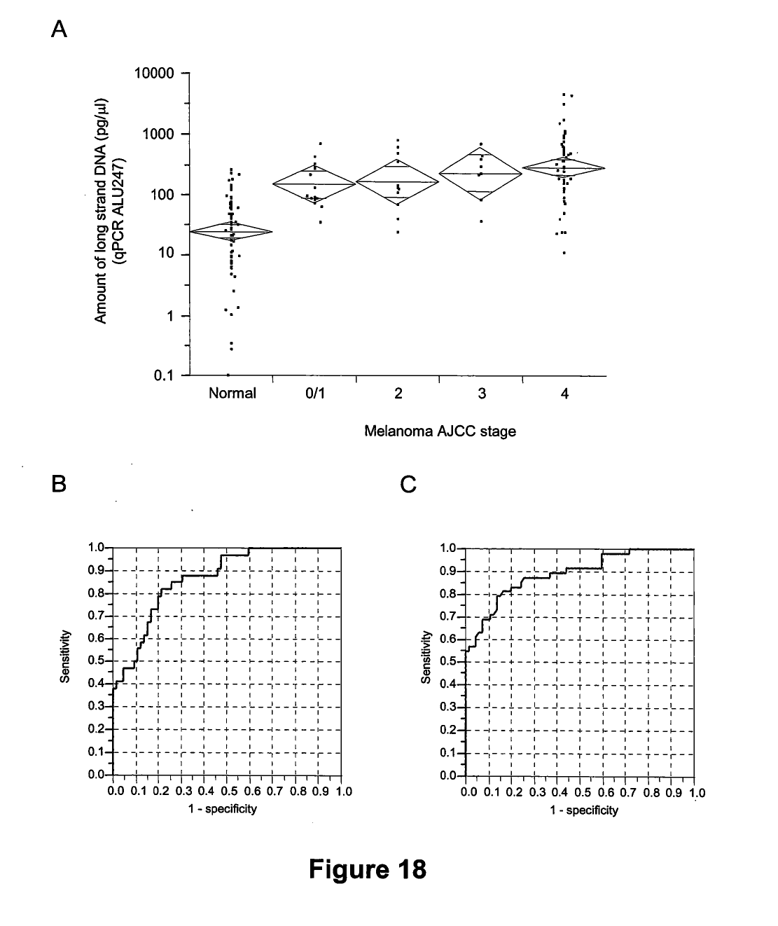

Because the amount of longer DNA fraction in serum represents the DNA released by non-apoptotic cell death, we quantified it with ALU247-qPCR. The mean logarithmic transformed values of longer DNA fraction (pg/μl) in normal controls and in AJCC stage 0/I, II, III, and IV melanomas were 1.40±0.08, 2.18±0.16, 2.22±0.19, 2.37±0.22, and 2.47±0.09, respectively (mean±SEM) (FIG. 18A). AJCC stage 0/I, II, III, and IV melanomas had significantly higher longer DNA fraction than in normal controls (P<0.0001, P<0.0001, P<0.0001, and P<0.0001, respectively). Receiver operating characteristic (ROC) curve for the existence of stage 0-III melanoma and stage IV melanoma according to the amount of the longer DNA fragment had an area under curve (AUC) of 0.87 and 0.89, respectively (FIGS. 18B and 18C).

### DNA Integrity of Circulating DNA

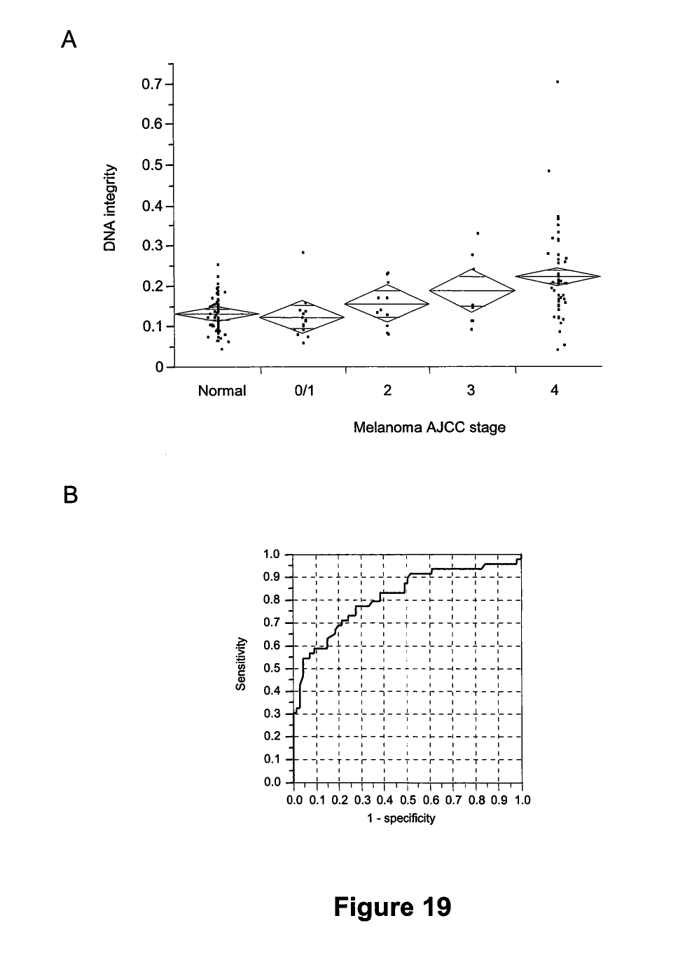

To assess the relative amount of long DNA fraction, DNA integrity was calculated in each specimens as (DNA integrity)=(qPCR result of ALU247)/(qPCR result of ALU115). Mean values of DNA integrity in normal controls and in AJCC stage 0/I, II, III, and IV melanomas were 0.13±0.01, 0.12±0.02, 0.16±0.02, 0.19±0.03, and 0.22±0.02, respectively (mean±SEM) (FIG. 19A). In patients with melanoma, DNA integrity was elevated with the progression of the disease (Spearman's ρ=0.47, P<0.0001). DNA integrity in stage I was almost the same as in normal controls, stages II and III had tendencies to have higher values of DNA integrity. Patients with stage IV melanomas showed significantly higher values of DNA integrity (P<0.0001). Receiver operating characteristic (ROC) curve for the existence of metastatic melanoma by the amount of the longer DNA had area under curve (AUC) of 0.82 (FIG. 19B).

### Discussion

The amount of the long DNA, quantified with ALU247-qPCR, represents the DNA released from non-apoptotic cells, which is unusual in healthy individuals. Therefore, it is a promising biomarker for detection of certain kinds of diseases such as malignant tumors. In contrast, the qPCR result of ALU115 (the short fragment DNA) represents the total amount of the circulating DNA, including DNA released from both apoptotic cells and non-apoptotic cells. Therefore, this value can be influenced by not only abnormal conditions, but also vital status of the donors, such as their basal metabolism rate. The DNA integrity, the ratio of the longer fragment DNA to the shorter fragment DNA, represent the relative amount of non-apoptotic cell death over the total cell death in the body.

Several methods of quantifying the amount of the DNA were used in the previous studies assessing circulating DNA in melanoma patients. Usually, the recovery rate and size distribution of the DNA from serum highly depends on the extraction method. For example, DNA affinity column methods usually lose short fragment DNA less than 100-200 bp. Therefore, quantifying DNA released from apoptotic cells is inaccurate with the column method. In addition, the quantification of DNA is difficult with limited amount of DNA available. Recent methods utilizing quantitative PCR of a specific locus of a gene achieved higher sensitivity, but allelic imbalance in cancer cells may affect the results and therefore the results can be inaccurate. To resolve these problems, we developed a novel method of direct qPCR on ALU repeats. Because ALU repeats are widely distributed in the human genome, their copy numbers are constant even in chromosomal instable malignant cells. Furthermore, because the high copy number drastically increases the efficiency of PCR, the template needed for the reaction is very low. As a result, 0.1 μl of serum is sufficient for quantifying the amount of DNA, and PCR using serum directly as a template without extracting DNA is made possible. The effect of inhibitory substances in serum has been taken into account and the qPCR results still have shown linearity in the plot without fluctuation. The error of the qPCR values contributed by the inhibitory substances is negligible, since it falls below the error limits of qPCR itself. In addition, by utilizing two sets of primers whose amplicon sizes were 115 bp and 247 bp, respectively, the size distribution of the circulating DNA can be measured accurately. Therefore, we consider that our novel method, the direct qPCR on ALU repeats, is ideal for the assessment of circulating DNA in patients.

Melanoma patients had significantly higher amount of long DNA fragment in their serum, independent of the stage of the disease. The tumor size in stage I melanomas is small and it is unlikely that the elevated serum DNA is released from only the tumor cells. The melanoma patients may have a constitutional characteristics of having a high level of circulating DNA, but further investigations will be needed to elucidate the mechanism. Despite of the reason, the amount of long DNA fragment in the serum is demonstrated to be a good diagnostic biomarker for malignant melanoma.

In contrast, patients with stage I melanoma had equivalent DNA integrity value as normal controls. It means that the circulating DNA is dominantly short fragment DNA released from apoptotic cells. However, patients with stage IV melanomas showed significantly higher DNA integrity than normal controls, demonstrating that the percentage of DNA released from non-apoptotic cells was higher in the advanced stage melanoma. This phenomenon can be reasonably explained. One reason is that, in advanced melanoma, hypoxic necrosis may occur in a hypovscular metastatic mass. Another reason is that melanoma cells in metastatic mass or shed into the bloodstream were attacked by immune cells such as natural killer cells and were ruptured and released DNA. Therefore, DNA integrity that represents the difference of cell death is a promising biomarker for detecting advanced melanomas. It would be useful for regular check-ups in the postoperative follow-up of melanoma patients.

In conclusion, we developed a highly sensitive method for DNA quantification, with lower detection limit of only 0.01 pg of DNA, equivalent to 1/300 copy of a human genome. It resulted in accurate and reproducible quantification of circulating free DNA in only 0.1 μl of serum by direct qPCR without extraction steps. Using this novel method, we established a method for melanoma detection based on the fact that sera from patients with malignancies have a higher amount of long fragment DNA or DNA integrity than those from healthy individuals. Retrospective assessment of sera drawn from preoperative patients with melanomas revealed that the amount of long fragment DNA is useful as a detection marker for early or advanced stages of melanomas. DNA integrity was elevated in patients with melanomas in stage-related fashion. It was significantly higher in patients with stage IV melanomas and was useful as detection markers for melanoma recurrence. This inexpensive method to measure DNA indices has potential as a practical detection tool for melanoma in regular checkups or in postoperative follow-ups.

### EXAMPLE V

### LINE1 and Unmethylated LINE1

### Introduction

We have developed novel, ultrasensitive assays to detect genotypic markers in acellular blood (serum) as diagnostic and prognostic markers for PCa (prostate cancer). We have shown that tumor-specific double and single stranded DNA can be detected in the serum/plasma of various cancer types using highly sensitive and informative PCR multimarker assays (2-5,36). These studies break the traditional paradigm that is based upon the belief that tumor-specific DNA can only be assessed in tumor tissue. The prognostic role of circulating DNA in serum/plasma was first demonstrated by our laboratory and has subsequently been confirmed by others (3). We have demonstrated that the multimarker approach of assessing mRNA and DNA markers in serum/plasma is more efficient than single markers in addressing tumor heterogeneity (1,3-4,36,63-64).

Molecular detection studies of circulating PCa tumor cells in blood using PSA (prostate-secific antigen) mRNA markers have been controversial, with inconsistent results between laboratories. Detection of circulating DNA offers a more stable biomarker with the potential for high-throughput quantitative utility. There are several major types of circulating DNA biomarkers, including microsatellites, methylation of promoter regions, and mutations. Several of these assays are now being validated in other cancers. In PCa, however, they are limited. Individual assays for specific genomic aberrations have been developed in our laboratory to assess various cancer patients' blood and were shown to correlate with stage, clinical pathology, prognosis, and disease recurrence (1,3-4,36). In PCa, the frequency of a specific LOH (loss of heterozygosity) marker in serum is not always concordant with that of the frequency in tumor tissue, which limits the utility of LOH as a serum biomarker. Non-informative results (i.e., homozygosity), further limit LOH marker assays for PCa. There is evidence for multifocality of prostate tumor development, as well as intra-and inter-tumoral heterogeneity which compounds the problem of using a single tumor marker for any one particular gene (64-67). Methylation of promoter regions has been observed in specific tumor-related genes in PCa during progression (65,68-71), but the detection of these biomarkers in plasma/serum is limited by variable methylation rates in early and late stages of PCa progression and poor overall detection in blood. A consistent serum PCa genotypic marker that is not significantly influenced by tumor heterogeneity or phenotype is needed.

The mechanism of the release of tumor DNA in blood is still unknown. It is believed that cancer cells that succumb to apoptotic-related mechanisms release DNA fragments. Lo et al. (9-10) demonstrated that EBV (Epstein-Barr virus) DNA liberated in the plasma/serum of patients with nasopharyngeal carcinoma reflects tumor cell death induced by radiotherapy, and that early recurrences were marked by a higher residual median concentration of tumor-associated DNA following treatment. These studies demonstrate the potential of detecting circulating tumor-derived DNA as a prognostic variable and as a tool for monitoring tumor response to various treatment modalities, including radiotherapy (10). These studies have opened up new approaches for understanding the in vivo effects of radiation at a molecular level. DNA markers may be developed as surrogates to assess responsiveness of PCa to radiotherapy. Tumor-related DNA markers may serve to monitor the kinetics of tumor cell death during radiation treatment. Surgical staging remains one of the most accurate methods of assessing patients' disease status. The assessment of blood before and after surgery provides a unique opportunity to evaluate disease burden relative to the presence of tumor-related DNA in the blood. The hypothesis is that if the tumor is removed, serum tumor-related DNA positive patients (pre-surgery) should have a decrease or elimination of serum tumor-related DNA. We will investigate the effect of surgery alone and in conjunction with radiotherapy on the integrity of serum tumor-related DNA.

The DNA biomarker assays described here provide a unique approach to take advantage of DNA integrity products resulting from tumor development and progression. Our strategy is to use specific repetitive sequences of the human genome as genomic biomarkers. Specifically, we will take advantage of the integrity of these genomic biomarkers for PCa detection and determinants of tumor progression. We will employ a multimarker strategy to develop a more sensitive assay that addresses tumor heterogeneity.

The majority of the human genome consists of repetitive sequences that include large segments of duplications, interspersed transposon-derived repeats and tandem repeats (27,72-73). These repetitive sequences are interspersed between and within coding and regulatory sequences. It is estimated that >50% of the human genome derives from transposable elements that include SINEs (short interspersed nuclear elements) and LINE1s (long interspersed nuclear elements 1). Of these transposable elements, the SINEs of primate-specific ALU sequences are the most abundant and account for >10% of the human genome (72-75). ALU, non-coding regions, are short (approx. 300 bp) GC-rich, non-autonomous elements characterized by the ALU endonuclease recognition sequence, 5′-AG/CT-3′. LINE1s exist as long (>6 kb) GC-poor sequences that are truncated at the 5′-end and can be transcribed into RNA, reverse transcribed into cDNA or reintegrate into the genome as cDNA. They represent about 17% of the human genome, and are referred to as class II retrotransposons. LINE1s may act as molecular rheostats, regulating gene expression (13,76-77). ALU 3′-ends are necessary for LINE1 transposition (73,78). We observed that LINE1 and ALU integrity (size) in serum can be detected as circulating DNA and are therefore potential serum biomarkers. The circulating DNA integrity in serum can be an indicator of non-apoptotic death. The DNA released from necrotic malignant cells varies in size, whereas DNA released from apoptotic cells is uniformly truncated to 120-200 bp. Because the main source of free circulating DNA in healthy individuals is apoptotic cells, a preponderance of longer fragments of DNA is a marker of malignant tumor cells (4,36). Repetitive genomic sequences such as SINEs and LINE 1s are an excellent source of DNA released by tumor cells. However, the integrity of free circulating DNA repeats has not been practical in the past due to the lack of specific, sensitive and quantitative assays. A major problem is the inability to obtain high yields of DNA from serum/plasma. A direct serum assay for these repeat genomic sequences was successfully developed in our laboratory.

Another important aspect of LINE1s is that methylation of CpG islands sites in the LINE1 promoter region are important for maintaining transcriptional inactivation and repressing retrotransposition of LINE1 elements (77,79-80). In general, normal tissue, CpG islands of LINE1 promoter regions are heavily methylated; demethylation leads to transcription of LINE1 elements (77,79-80). Retrotransposition of LINE1 can lead to inactivation of tumor suppressor genes or to the activation of oncogenes when placed next to a promoter region. The hypomethylated phenotype of genomic sequences has been shown to relate to chromosome instability and tumor progression (71,80-81), which we have recently demonstrated in gastric and colorectal carcinoma. The unmethylated LINE1 (uLINE1) promoter regions may be valuable biomarkers; their location on all chromosomes may overcome problems resulting from tumor heterogeneity. We developed an assay to detect uLINE1 as a surrogate of tumor progression; more aggressive tumors will have increased genetic instability and higher levels of hypomethylation.

Recently, we developed a simple, robust, highly-sensitive, and high-throughput method to assess free circulating DNA in serum. We will optimize and validate a highly sensitive DNA biomarker assay to assess ALU and LINE1 integrity in the sera of PCa patients, taking advantage of the types of DNA repetitive sequences (GC-rich vs GC-poor) (41,82-83). In addition, we will assess uLINE1 as these unmethylated markers are abundant in serum and stay intact during release from tumor cells. This is a valuable surrogate biomarker in serum to assess tumor genetic instability. The detection of these specific DNA markers in serum can together be used to identify patients with disease progression and tumors likely to be aggressive or fatal to patients. The initial plan is to determine the efficacy of these DNA biomarkers alone and in combination with known prognostic factors such as PSA.

A major limitation of determining the success of PCa treatment is the accuracy of assessing residual disease and tumor progression. It is clear that new markers are needed beyond serum PSA to improve the assessment of patients' risk and response to treatment. Approximately 30% of men with pathologically organ-confined disease experience an early relapse despite successful treatment of the primary lesion. In addition, approximately 50% of men with clinically organ-confined lesions are found to be understaged at the time of surgery (61-62). The problem of understaging patients is a major concern and needs to be better addressed with alternative non-invasive procedures that can be repetitively assessed.

### Results

The feasibility of detecting circulating fragments of repetitive sequences such as ALU, LINE1 and uLINE1 in PCa serum has been demonstrated recently in our laboratory. We have performed pilot studies to suggest that these genomic biomarkers increase with tumor progression in PCa patients' serum. The studies are high risk, but if successful, will break traditional paradigms in diagnosing and monitoring PCa progression. We have developed all three genomic biomarkers.

### Detection of Circulating DNA in Serum/Plasma and its Prognostic Role in Cancer Patients

Our laboratory has pioneered translational studies involving the detection, prognostic importance and predictive utility of circulating DNA in cancer patients' serum. We have developed reproducible techniques of isolating DNA from plasma/serum (4,17,36,48,84) and have demonstrated the utility of monitoring for DNA before, during and after therapy (5,36). Circulating DNA biomarkers provide a unique opportunity to monitor tumor genetic changes serially without sampling the tumor. Our laboratory has demonstrated that using multiple DNA markers is far more effective than single markers (1,5).

### Analysis of Microsatellite for LOH in PCa Patients' Sera

Previously, we detected several LOH markers in PCa patients' serum using capillary array electrophoresis (CAE). Microsatellite markers for LOH were detected in sera using four markers on chromosomes 9, 10, 16 and 18 in AJCC stage I-IV PCa patients. No markers were detected in normal healthy male donor sera (n=25). In 8 informative stage I & II patients' sera, 2 (25%) were LOH positive; in stage III & IV, 31% were LOH positive. The assay is limited because not all patients are informative for a particular microsatellite marker. Furthermore, this assay is not quantitative. We have also developed assays to assess methylation of CpG promoter regions and other regulatory elements of a variety of genes using CAE, including GSTP1, RASSF1A, and RARβeta 2. GSTP1, RASSF1A and RARβeta 2, frequently methylated at CpG sites in their promoter regions in PCa tissues. Preliminary results on PCa cancer patients' sera demonstrated that no stage I & II patients showed methylated markers, whereas 50% of 16 stage III & IV patients tested showed methylated markers. The optimal gene combination is unknown and no individual methylation marker can be detected in >90% of tumors. Another major problem with microsatellite and methylation markers is their relatively low frequency in early stage disease. Nevertheless, circulating tumor-related DNA was detectable in the blood of PCa patients and not in normal donors, and there was a correlation with disease progression. A more consistent set of DNA markers is needed to improve the sensitivity of serum DNA biomarker assays. We propose using the repetitive non-coding DNA sequences such as ALU and LINE1 to address the above problems.

Direct qPCR Assay for Detection of ALU Repeats in Prostate Cancer Patients' Sera

Recently, we have developed a direct quantitative real-time PCR (qPCR) assay for assessment of DNA integrity in serum to assess different sizes of ALU fragments. The direct assay allows for PCR without prior DNA extraction. This was successfully developed and assessed on >200 cancer patients and >100 normal donors.

Blood for circulating DNA assays is collected in a tiger tube and processed within 2-4 hrs. The tiger tubes are centrifuged and serum is then collected and filtered (1Ou pore) to remove extraneous cells. The filtered serum (50 μl ) is combined with a mixture containing Proteinase K (Qiagen) and a specific preparation PCR buffer. After vortexing, the sample is incubated at 50° C. for 20 min to allow for protein digestion. Immediately afterward, the samples are incubated for 5 min at 95° C. to denature the proteins, then centrifuged at room temperature. Finally, the supernatant is removed and diluted with TE buffer (pH 8.0) prior to qPCR.

For this study, we determined that long serum DNA fragments are released from necrotic tumor cells and are not truncated before release, whereas DNA released from normal apoptotic cells is truncated to 120-200 bp fragments. To detect the longer pieces of DNA, primers were used to amplify a 247 bp amplicon of the ALU sequences. Other primers were created to detect both truncated and shorter fragments by amplifying a 115 bp amplicon. PCR reactions are carried out using a qPCR detection system (ABI 7900HT). The samples are run in triplicate and the DNA standards are run in duplicate. The standards consist of serially diluted known concentrations of ALU sequences. As controls, 3 positive and 3 normal donor specimens (various levels of ALU) with a known DNA integrity are run on every plate.

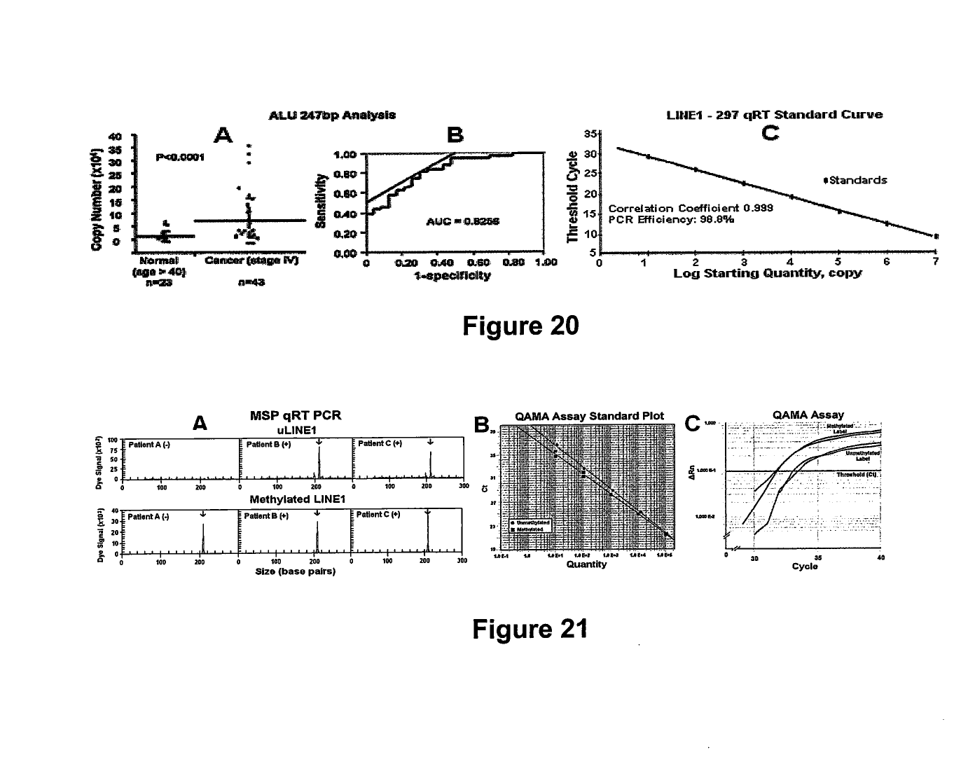

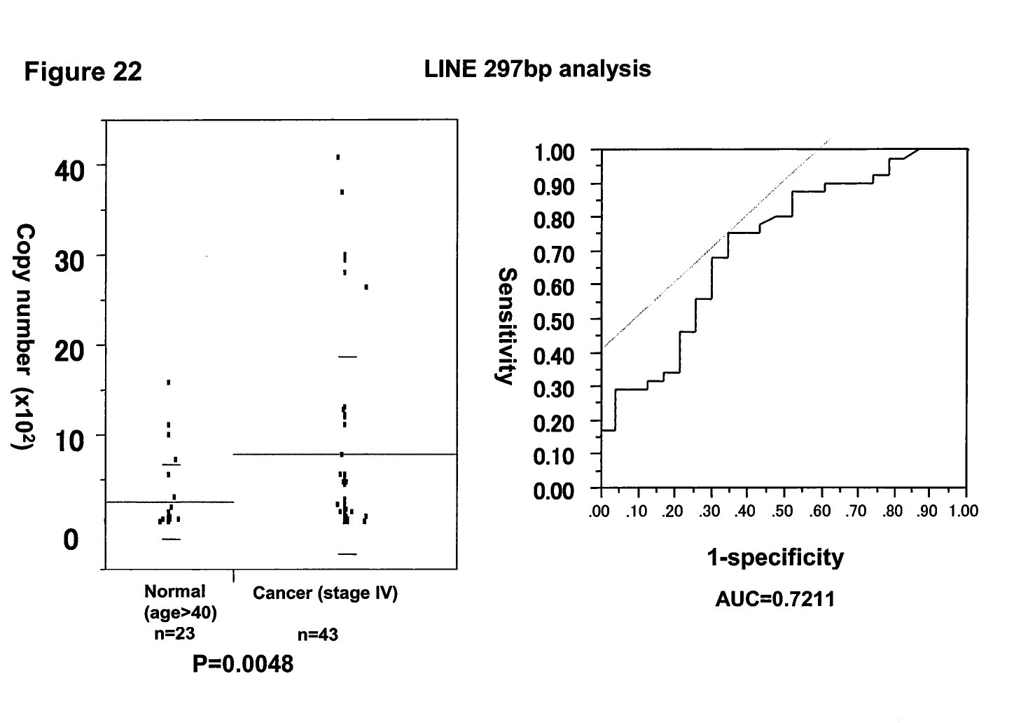

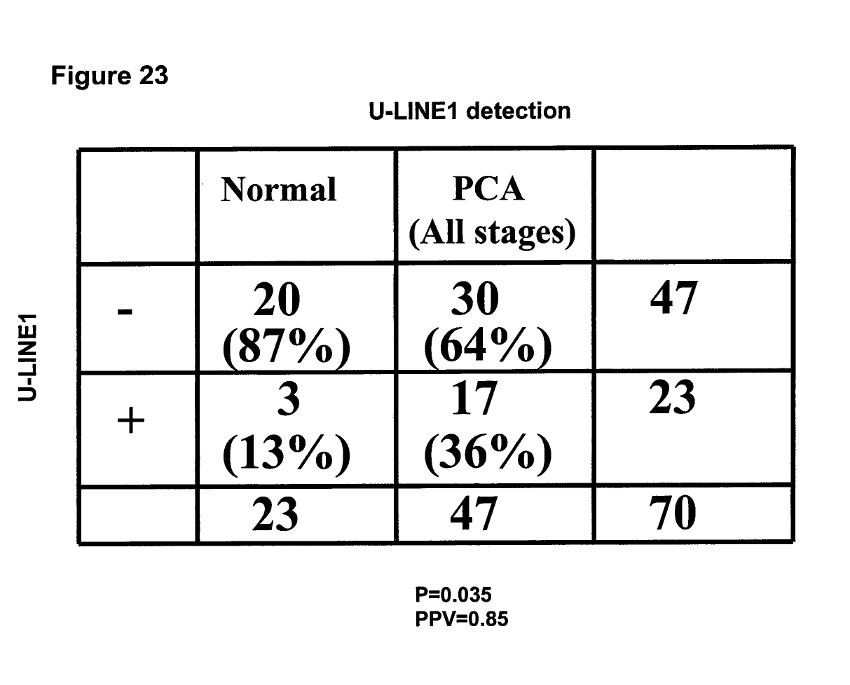

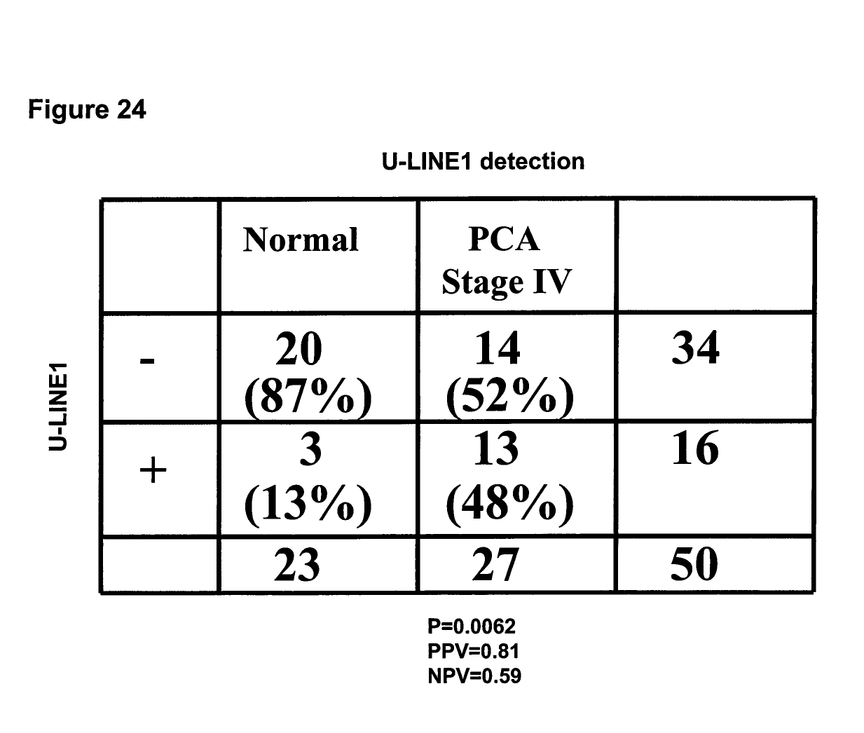

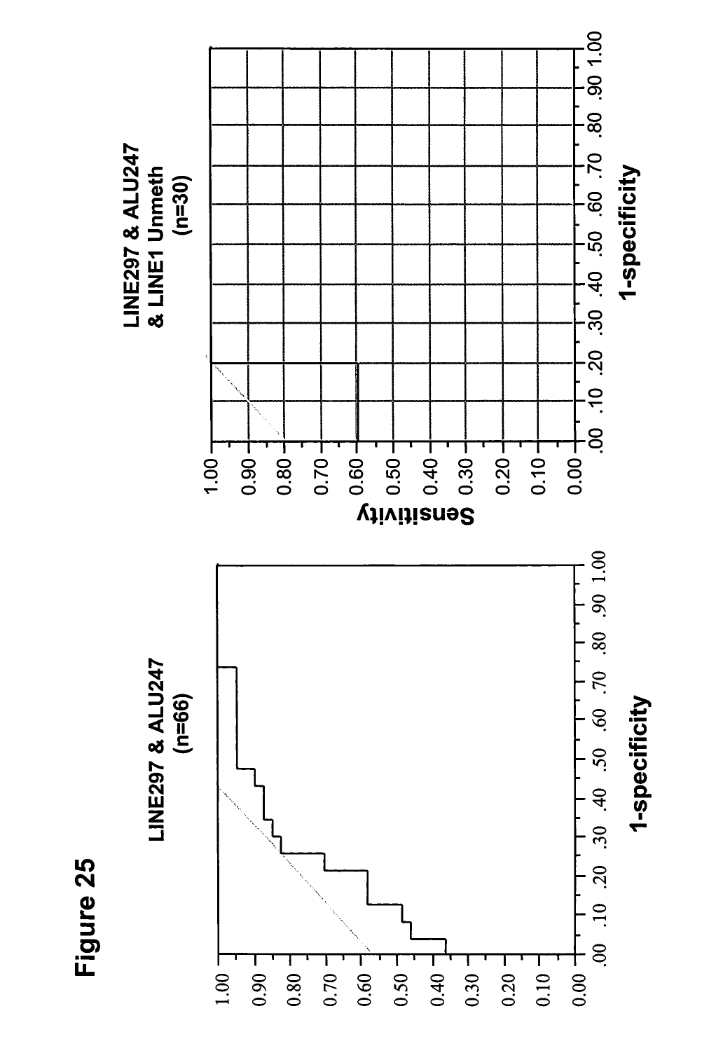

In preliminary studies, we performed a direct qPCR for ALU247 in PCa patients and normal healthy donors. The results were highly encouraging, demonstrating the feasibility, sensitivity and potential clinical utility of the approach. Comparing AJCC stage IV PCa patients (n=43) to normal healthy male donors (>40 yrs), we demonstrated a significantly higher ALU247 copy number in PCa patients (P<0.001; FIG. 20A). In plotting a ROC (receiver operating curve), the area under the curve (AUC) was 0.83 (FIG. 20B).

### Detection of LINE1 Repeats in PCa Patients' Sera

A direct qPCR assay to assess large fragments of LINE1 297 was developed (FIG. 20C). The size of the PCR products represents a DNA integrity fragment that is likely due to the non-apoptotic release of tumor cells. Different fragment sizes were also assessed in a similar fashion as ALU to determine optimal detection and informative size. Serum was prepared as for the ALU-qPCR described above. Compared to stage IV AJCC patients (n=43), normal healthy male donors (>40 yrs) had significantly (P<0.005) lower copy numbers of LINE1. In plotting an ROC, the AUC was 0.72. The assay has been very encouraging in pilot studies, demonstrating potential clinical utility.

Detection of uLINE1 in Prostate Cancer Patients' Sera

In preliminary studies, we assessed methylated and uLINE1 by semi-quantitative CAE. DNA from 500 ul of serum was extracted and subjected to sodium bisulfite modification as previously described (36). This procedure has now been modified and streamlined using a Qiagen bisulfite modification kit to improve DNA yields and reduce time.

Methylation status of LINE1 was assessed using two sets of fluorescent labeled primers specifically designed to amplify methylated or unmethylated DNA sequences of the LINE1 promoter region. Bisulfite-modified DNA was subjected to PCR amplification with AmpliTaq. PCR amplification was performed using 35 cycles. Lymphocyte DNA underwent sodium bisulfite modification and a universal unmethylated control synthesized by phi-29 DNA polymerase from normal DNA served as a positive unmethylated control as previously described (84). Unmodified lymphocyte DNA was used as a negative control for methylated and unmethylated reactions. SssI Methylase-treated lymphocyte DNA was used as a positive methylation control. PCR products were visualized using CAE (CEQ 8000XL; Beckman Coulter, Inc). Methylated and unmethylated products from each sample were assessed by multiplexing using forward labeled primers with Beckman Coulter WellRED dye-labeled phosphoramidites. Forward methylated sequence-specific primers were labeled with D4pa dye, and forward unmethylated sequence-specific primers were labeled with D3a dye. Each marker was optimized with methylated and unmethylated controls. Only those samples demonstrating a peak at the specific corresponding by size for unmethylated DNA were considered as uLINE1 (FIG. 21A). In the pilot analysis of AJCC stage IV PCa patients (n=27) versus normal male healthy donors (n=23), there were significantly (P=0.006) higher values of uLINE1for the stage IV patients' sera (FIGS. 21B and 21C). The prognostic predictive value was 0.81. In the comparison of AJCC stage II-IV PCa patients (n=47) versus normal donors (n=23), there was a significant (P=0.035) difference; the prognostic predictive value was 0.85. Preliminary results were encouraging and suggested that uLINE1 can be used as a biomarker for PCa. When LINE1, ALU, and uLINE1 were assessed together, an AUC of 0.92 was found. For the combination of uLINE1 and ALU247, we obtained an AUC of 0.911 (n=30). These preliminary analyses demonstrate the potential clinical utility of combining the three DNA biomarkers.

Recently, we have developed a QAMA (quantitative analysis of methylated alleles) assay for the quantification of methylated and unmethylated products of the same allele (FIGS. 21B and 21C). The assay is qPCR-based and uses bisulfite treated DNA (85), using TaqMan probes based on minor groove binder (MGB) technology (ABI). It has significant advantages over the MethyLight assay (83). Both methylated and unmethylated alleles can be detected in the same reaction (FIG. 21C) and are each quantified by a respective standard curve (FIG. 21B). FIG. 21C shows an example of analysis repetitive genomic sequences methylated and unmethylated. The assay can distinguish CpG dinucleotide polymorphisms, and can be assessed with minimal inter-assay variation (85).

### Detection of LINE1 in Breast Cancer Patients' Sera

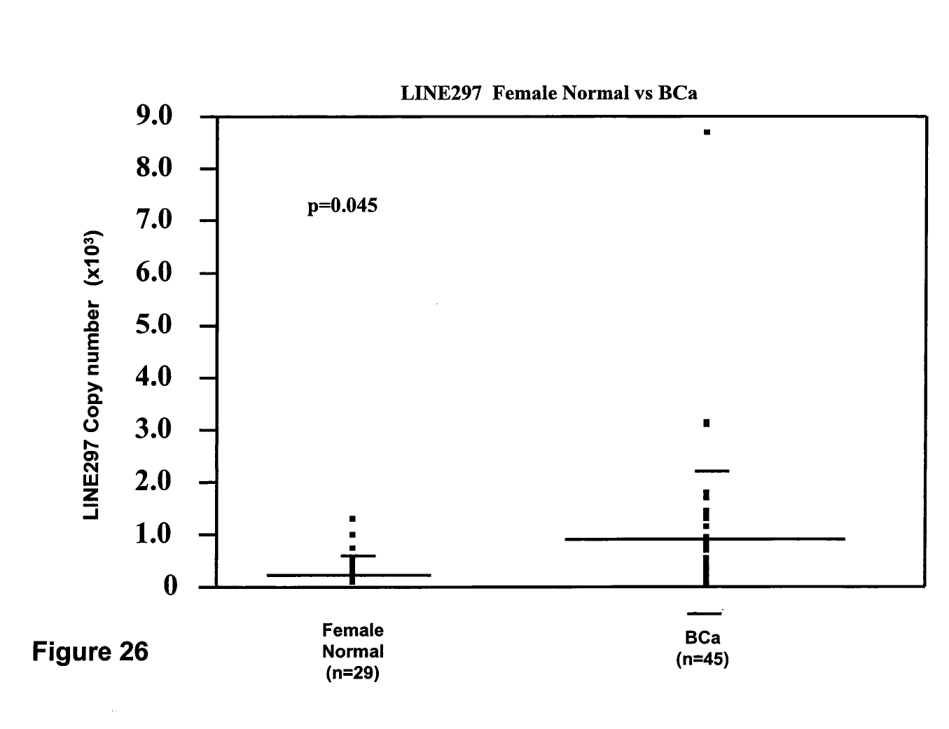

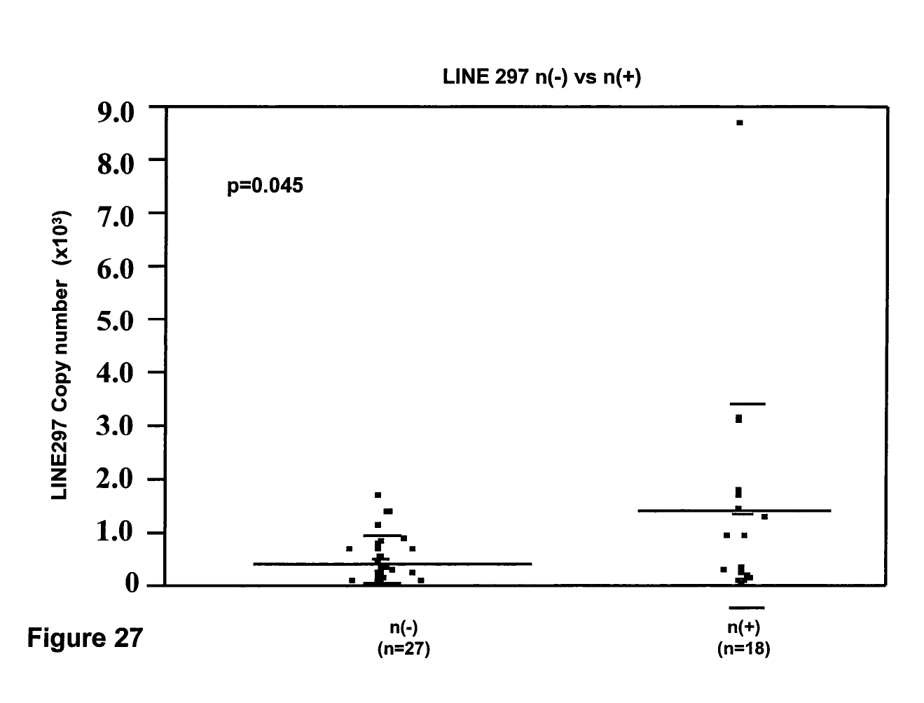

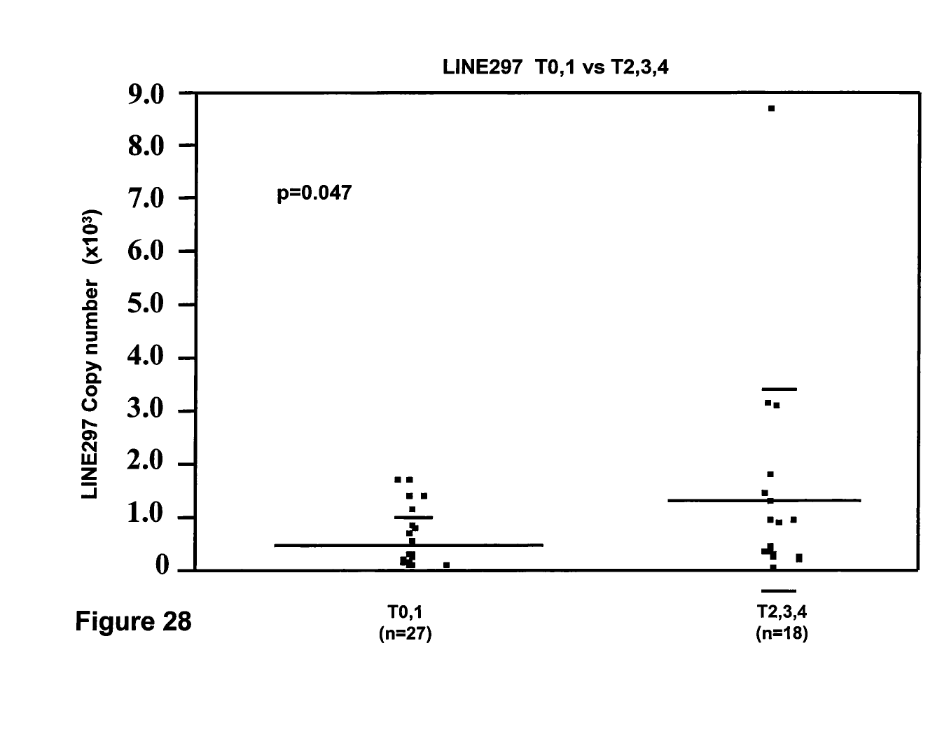

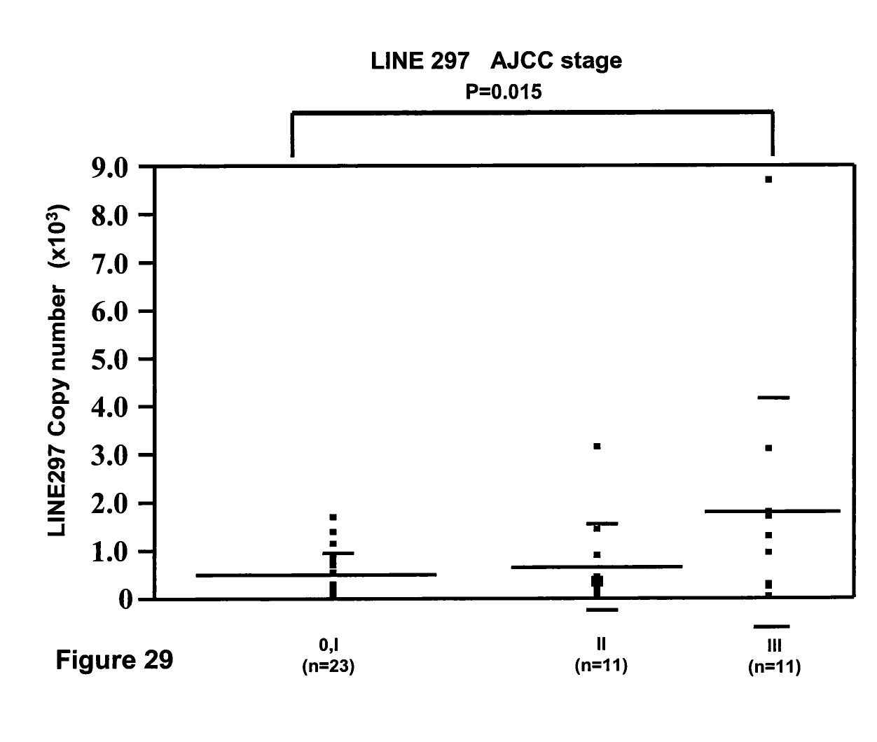

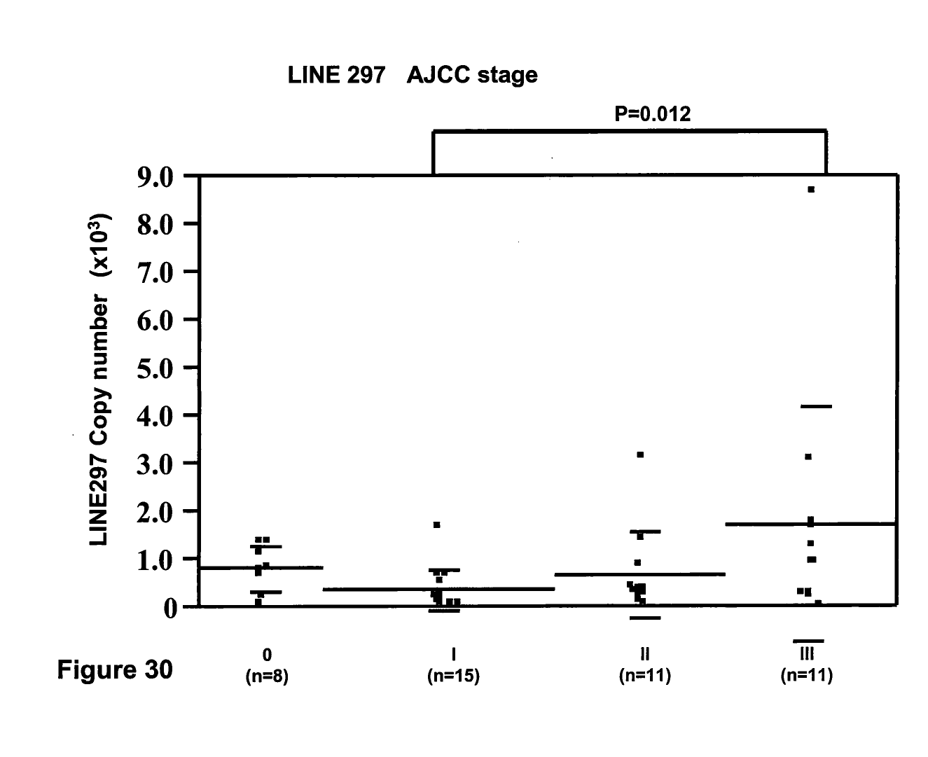

Absolute amount of long DNA fragment was quantified by qPCR with LINE1 297 primer set in normal group and in breast cancer groups. qPCR values of LINE1 297 were significantly higher in breast cancer patients than in normal group (FIGS. 26), in lymph node (LN) positive group than in LN negative group (FIG. 27), in T2 to T4 group than in T0 to T1 group (FIG. 28), and in stage III cancers than in stage 0 to I cancers (FIG. 29).

### REFERENCES

1. Fujiwara et al. (1999) Cancer Res 59:1567-71.
2. Taback et al. (2001) Ann NY Acad Sci 945:22-30.
3. Taback et al. (2001) Cancer Res 61:5723-6.
4. Taback and Hoon (2004) Curr Opin Mol Ther 6:273-8.
5. Taback et al. (2004) J Natl Cancer Inst 96:152-6.
6. Sorenson (2000) Clin Cancer Res 6:2129-37.
7. Stroun et al. (1989) Oncology 46:318-22.
8. Nawroz et al. (1996) Nat Med 2:1035-7.
9. Lo et al. (2000) Cancer Res 60:6878-81.
10. Lo et al. (2000) Cancer Res 60:2351-5.
11. Esteller et al. (1999) Cancer Res 59:67-70.
12. Nawroz et al. (1996) Nat Med 2:1035-7.
13. Chen et al. (1996) Nat Med 2:1033-5.
14. Hibi et al. (1998) Cancer Res 58:1405-7.
15. Mulcahy et al. (1998) Clin Cancer Res 4:271-5.
16. Anker et al. (1999) Cancer Metastasis Rev 18:65-73.
17. Taback and Hoon (2004) Ann NY Acad Sci 1022:1-8.
18. Fujimoto et al. (2004) Cancer Res 64:4085-8.
19. Taback et al. (2004) Ann NY Acad Sci 1022:17-24.
20. Gautschi et al. (2004) J Clin Oncol 22:4157-64.
21. Holdenrieder et al. (2005) Clin Chem 51:1544-6.
22. Chan et al. (2005) Clin Chem 51:781-4.
23. Gu et al. (2000) Gene 259:81-8.
24. Martin (2000) Clin Chem 46:100-4.
25. Giacona et al. (1998) Pancreas 17:89-97.
26. Hwu et al. (1986) PNAS USA 83:3875-9.
27. Smit (1996) Curr Opin Genet Dev 6:743-8.
28. Jemal et al. (2005) CA Cancer J Clin 55:10-30.
29. Fisher et al. (1983) Cancer 52:1551-7.
30. Sanchez-Cespedes et al. (2000) Cancer Res 60:892-5.
31. Gal et al. (2004) Br J Cancer 90:1211-5.
32. Silva et al. (2002) Clin Cancer Res 8:3761-6.
33. Fiegl et al. (2005) Cancer Res 65:1141-5.
34. Dulaimi et al. (2004) Clin Cancer Res 10:6189-93.
35. Muller et al. (2003) Cancer Res 63:7641-5.
36. Mori et al. (2005) J Clin Oncol. 23:9351-8.
37. Wang et al. (2003) Cancer Res 63:3966-8.
38. Wyllie (1980) Nature 284:555-6.
39. Jin and El-Deiry (2005) Cancer Biol Ther 4:139-63. Epub 2005 February 2027.
40. Jahr et al. (2001) Cancer Res 61:1659-65.
41. Lander et al. (2001) Nature 409:860-921.
42. Leon et al. (1977) Cancer Res 37:646-50.
43. Holdenrieder et al. (2004) Clin Cancer Res 10:5981-7.
44. Herrera et al. (2005) Clin Chem 51: 113-8. Epub 2004 November 2011.
45. Wagner et al. (2004) Ann NY Acad Sci 1022:9-16.
46. Umetani et al. (2006) Clin Chem 52: in press.
47. Jung et al. (2003) Clin Chem 49:1028-9.
48. Umetani et al. (2006) Ann NY Acad Sci, in press.
49. Frebourg et al. (1988) Cancer 62:2287-90.
50. Duffy (2001) Clin Chem 47:624-30.
51. Jurka (1998) Curr Opin Struct Biol 8:333-7.
52. Uemura et al. (2004) J Gastroenterol 39:56-60.
53. Lindforss et al. (2005) Anticancer Res 25:657-61.
54. Lam et al. (2003) Clin Chem 49:1286-91.
55. Chan et al. (2004) Clin Chem 50:88-92.
56. Winawer et al. (2003) Gastroenterology 124:544-60.
57. Mandel et al. (1993) N Engl J Med 328:1365-71.
58. Graham et al. (1998) Ann Surg 228:59-63.
59. Griffin et al. (1990) Cancer 66:56-61.
60. Westerdahl et al. (1993) Hepatogastroenterology 40:384-7.
61. Solokoff and Brendler (2000) Radical retropubic prostatectomy. In: Devita et al. (eds.) Cancer: Principles & Practice of Oncology, 6th edition, Vol. 14, pp. 1-15. New York: Lippincott-Raven.
62. Bostwick and Foster (1999) Semin Urol Oncol 17:222-72.
63. Bostick et al. (1999) J Clin Oncol 17:3238-44.
64. Isaacs and Bova (2002) Prostate Cancer. In: Vogelstein and Kinzler (eds.) The Genetic Basis of Human Cancer, 2nd edition, pp. 709-718. New York: McGraw-Hill Companies, Inc.
65. Konishi et al. (2002) Am J Pathol 160:1207-14.
66. Hugel and Wernert (1999) Br J Cancer 79:551-7.
67. Macintosh et al. (1998) Cancer Res 58:23-8.
68. Maruyama et al. (2002) Clin Cancer Res 8:514-9.
69. Goessl et al. (2000) Cancer Res 60:5941-5.
70. Millar et al. (1999) Oncogene 18:1313-24.
71. Schulz et al. (2002) Genes Chromosomes Cancer 35:58-65.
72. Jasinska and Krzyzosiak (2004) FEBS Lett 567:136-41.
73. Pavlicek et al. (2001) Gene 276:39-45.
74. Jurka (2004) Curr Opin Genet Dev 14:603-8.
75. Kolomietz et al. (2002) Genes Chromosomes Cancer 35:97-112.
76. Feng et al. (1996) Cell 87:905-16.
77. Nolan et al. (2005) Nucleic Acids Res 33:1564-73.
78. Zingler et al. (2005) Genome Res 15:780-9.
79. Burden et al. (2005) J Biol Chem 280:14413-9.
80. Roman-Gomez et al. (2005) Oncogene 24:7213-23.
81. Takai et al. (2000) Jpn J Clin Oncol 30:306-9.
82. Dewannieux et al. (2003) Nat Genet 35:41-8.
83. Weisenberger et al. (2005) Nucleic Acids Res 33:6823-36.
84. Umetani et al. (2005) Biochem Biophys Res Commun 329:219-23.
85. Zeschnigk et al. (2004) Nucleic Acids Res 32:e125.

1. A method of quantifying circulating human genomic DNA, comprising
amplifying an Alu repeat sequence in a body fluid sample from a subject without performing a DNA purification or extraction step, wherein the Alu repeat sequence is part of an Alu consensus sequence; and
quantifying an amount of the amplified Alu repeat sequence in the sample.

2. The method of claim 1, wherein the Alu repeat sequence is (i) 115 base pairs in length (ALU115) or (ii) 247 base pairs in length (ALU247).

3. The method of claim 1, wherein the body fluid sample is a blood, plasma or serum sample.

4. The method of claim 1, wherein the Alu repeat sequence is amplified by quantitative real-time polymerase chain reaction (qPCR).

5. The method of claim 1, wherein the amount of amplified Alu repeat sequence is quantified by determining an absolute equivalent amount of genomic DNA using a standard curve.

6. The method of claim 2, further comprising:
amplifying a second Alu repeat sequence in the body fluid sample without performing a DNA purification or extraction step, wherein the second Alu repeat sequence is 247 base pairs in length (ALU247);
quantifying an amount of the second amplified Alu repeat sequence (ALU247) in the sample; and
determining DNA integrity by calculating a ratio of ALU247 to ALU115.

7. A method of detecting malignant cancer in a subject comprising:
amplifying an ALU115 repeat sequence and/or an ALU247 repeat sequence in a body fluid sample from a subject suspected of having a malignant cancer without performing a DNA purification or extraction step;
quantifying an amount of the ALU115 and/or an amount of the ALU247 repeat sequences in the sample; and
determining that
(i) the subject likely has malignant melanoma or prostate cancer if the amount of ALU247 is higher in the subject than in a healthy individual; or
(ii) the subject likely has malignant colorectal cancer, periampullary cancer or breast cancer if a ratio of the amount of ALU247 to the amount of ALU115 (ALU247/ALU115) is higher in the subject than in a healthy individual.

8. The method of claim 7, wherein the body fluid sample is a blood, plasma or serum sample.

9. The method of claim 7, wherein the ALU115 and ALU247 repeat sequences are amplified by quantitative real-time polymerase chain reaction (qPCR).

10. The method of claim 7, wherein the amount of amplified Alu repeat sequence is quantified by determining an absolute equivalent amount of genomic DNA using a standard curve.

11. The method of claim 7, further comprising amplifying a LINE1 297 sequence in the body fluid sample;
quantifying an amount of the LINE1 297 sequence; and
determining that the subject likely has (i) malignant breast cancer or (ii) malignant or metastatic prostate cancer if the amount of LINE1 297 sequence is higher in the subject than in a healthy individual.

12. The method of claim 7, further comprising determining a level of unmethylated LINE1 (uLINE1) in the body fluid sample, and determining that the subject likely has malignant or metastatic prostate cancer if the amount of uLINE1 is higher in the subject than in a healthy individual.

13. The method of claim 12, wherein the level of uLINE1 is detected by quantitative analysis of methylated alleles (QAMA).

14. A method of monitoring progression of a cancer in a subject comprising:
amplifying an ALU115 repeat sequence and a ALU247 repeat sequence in a body fluid sample from a subject suspected of having a malignant cancer without performing a DNA purification or extraction step;
quantifying an amount of the ALU115 and an amount of the ALU247 repeat sequences in the sample; and
determining that the subject likely has a metastatic melanoma or breast cancer if a ratio of the amount of ALU247 to the amount of ALU115 (ALU247/ALU115) is higher in the subject than in a healthy individual or in a subject with a stage I melanoma or breast cancer.

15. The method of claim 14, wherein the body fluid sample is a blood, plasma or serum sample.

16. The method of claim 14, wherein the ALU115 and ALU247 repeat sequences are amplified by quantitative real-time polymerase chain reaction (qPCR).

17. The method of claim 14, wherein the amount of amplified Alu repeat sequence is quantified by determining an absolute equivalent amount of genomic DNA using a standard curve.

18. The method of claim 14, further comprising amplifying a LINE1 297 sequence in the body fluid sample;
quantifying an amount of the LINE1 297 sequence; and
determining that the subject likely has metastatic breast cancer if the amount of LINE1 297 sequence is higher in the subject than in a healthy individual.
